(19) United States
(12) Patent Application Publication
(10) Pub. No.: US 2007/0117861 A1
(43) Pub. Date: May 24, 2007

(54) Treatment or prevention of cardiovascular and respiratory disorders with novel substituted cyclic-amp specific phosphodiesterase inhibitors

(75) Inventors: Desuo Wang, Lexington, SC (US); Joseph W. Kosh, Lexington, SC (US); J. Walter Sowell, West Columbia, SC (US); Ting Wang, Columbia, SC (US)

Correspondence Address: NELSON MULLINS RILEY & SCARBOROUGH, LLP, 1320 MAIN STREET, 17TH FLOOR, COLUMBIA, SC, 29201, US

(73) Assignee: University of South Carolina, Columbia, SC (US)

(21) Appl. No.: 10/578,951

(22) PCT Filed: Nov. 12, 2004
(86) PCT No.: PCT/US04/37813

§ 371 Date: May 10, 2006

Related U.S. Application Data
(60) Provisional application No. 60/519,140, filed on Nov. 12, 2003.

(51) Int. Cl. A61K 31/401

(52) U.S. Cl. 514/423; 548/537

(57) ABSTRACT

Compositions and methods are described for the prevention and/or treatment of cardiovascular and respiratory disorders, the method comprising administering to the subject a novel cAMP-specific PDE inhibitor. Also described are therapeutic compositions, pharmaceutical compositions and kits that are useful in the present invention.

### CROSS-REFERENCE TO RELATED APPLICATIONS

The present application claims the benefit of U.S. Provisional Application Ser. No. 60/519,140 filed Nov. 12, 2003, which is incorporated herein by reference thereto.

### STATEMENT REGARDING FEDERALLY SPONSORED RESEARCH OR DEVELOPMENT

The U. S. Government has a paid-up license in this invention and the right in limited circumstances to require the patent owner to license others on reasonable terms as provided for by the terms of grant No. 5 K2200367-02 awarded by the U.S. National Institutes of Health of the Department of Health and Human Services.

### BACKGROUND OF THE INVENTION

(1) Field of the Invention

The present invention relates to certain novel substituted pyrrole compounds, and also to methods of using such compounds for the prevention and/or treatment of cardiovascular and respiratory disorders.

(2) Description of the Related Art

A broad spectrum of cardiovascular and respiratory disorders has been recognized, many of which have overlapping and interacting etiologies. Two of the most widespread and prevalent of these disorders are hypertension and asthma.

Asthma is a respiratory disorder that is characterized by reversible airway obstruction, airway inflammation, and increased airway responsiveness (manifested as bronchoconstriction), due to a variety of irritating stimuli. Airway obstruction in asthma is due to a combination of factors including spasm of airway smooth muscle, edema of airway mucosa, increased mucus secretion, and cellular infiltration of the airway walls. Acute symptoms of asthma usually begin quite suddenly with wheezing episodes, coughing and shortness of breath.

In the United States, an estimated 12 million people suffer from asthma. During the ten-year period from 1982 to 1992, the prevalence of asthma increased from 34.7 to 49.4 per 1000 and the death rate increased 40%, from 13.4 to 18.8 per million. See The Merck Manual of Diagnosis & Therapy, Beers & Brakow, 17^{th} edition, Published by Merck Research Labs, Sec. 6, Chapter 68, Chronic Obstructive Airway Disorders, COPD (1999). Asthma is now the leading cause of hospitalization for children.

Bronchoconstriction is the primary symptom of many respiratory disorders, including, asthma. Bronchoconstriction is an airflow limitation resulting from contraction of the smooth muscle that envelops the bronchi and bronchioles. This airway contraction makes it very difficult for air to pass through the lungs and can lead to symptoms of wheezing, coughing, tightness of the chest, and breathlessness as a subject suffering from such a symptom tries to breathe. When the mucosa lining the airway passages is thickened by inflammation from an existing respiratory disorder, even a minor smooth muscle contraction can substantially narrow the airways and make breathing more difficult.

Cardiovascular disease is responsible for every 1 in 2.5 deaths in the United States, claiming more lives each year than the next five leading causes of death combined, those being cancer, chronic lower respiratory disease, accidents, diabetes, and influenza/pneumonia. In the United States, there is an average of one death every 33 seconds that is attributed to cardiovascular disease. See American Heart Association. Heart Disease and Stroke Statistics—2003 Update. Dallas, Tex.: American Heart Association; 2002.

Hypertension is the most common cardiovascular disorder and the management of hypertension is the leading indication for both visits to physicians and the use of prescription drugs in the United States. Hypertension is closely associated with high morbidity, disability, and mortality from coronary heart disease and strokes. Although antihypertensive therapy can effectively prevent hemorrhagic strokes, cardiac failure, and renal insufficiency, which are due to high blood pressure, epidemiological studies demonstrate that only 27 percent of hypertensives had their blood pressure well controlled. As a result, the ongoing effort to discover new and improved antihypertensive agents remains one of the major challenges of modem pharmaceutical research and development.

The clinical treatment of hypertension involves various medicaments that lower overall blood pressure and prevent the cardiovascular complications that are known to accompany elevated blood pressure. Currently, five major classes of drugs are used to lower blood pressure, including diuretics, adrenergic inhibitors, calcium channel blockers (CCBs), renin-angiotensin inhibitors, and vasodilators. Currently, vasodilators, together with CCBs and angiotensin inhibitors, are the accepted choice as first line medicaments for controlling blood pressure.

CCBs were introduced into clinical medicine in the 1960s and are now among the most frequently prescribed drugs for the treatment of cardiovascular disorders. Although the currently available CCBs are chemically diverse, they share the common property of preventing the transmembrane flow of calcium ions through voltage-gated L-type (slowly inactivating) channels.

In both vascular and cardiac tissue, muscle cell contraction occurs when cells are depolarized from the influx of calcium through voltage-sensitive calcium channels in the cell. The increased cytosolic calcium binds to calmodulin, activating myosin light-chain kinase which phosphorylates myosin. The phosphorylated myosin can then interact with actin, resulting in muscle contraction.

CCBs work by blocking voltage-sensitive calcium channels in the cardiac muscle of the heart and in the smooth muscle surrounding blood vessels. This causes blood vessel walls to relax and blood to flow more freely to the heart. All CCBs improve coronary blood flow and are effective antianginal drugs. CCBs also act on the heart to improve filling by promoting relaxation of cardiac muscle during diastole. Since cardiac and smooth muscle contraction is dependent on calcium influx through slow channels, all CCBs relax vascular smooth muscle in both the peripheral and coronary circulation, but do not act on skeletal muscle.

In cardiac and vascular/airway smooth muscles, it is well established that the two most extensively studied G protein-coupled receptor (GPCR) systems, βadrenergic and muscarinic M₂ receptors, increase and decrease the cAMP signaling by activating and inhibiting adenylyl cyclase activity, respectively. Recent studies show that cAMP-specific phosphodiesterase enzymes (PDEs) act as a negative feedback regulator in protein kinase A (PKA) signaling and a necessary mediator in the muscarinic stimulation-caused airway constriction. Thus, cAMP-specific PDEs are critical in for the control and regulation of cAMP signaling.

PDEs comprise a large family of enzymes that catalyze the hydrolysis of the intracellular second messenger cyclic nucleotides, cAMP and cGMP, to their biologically inactive forms, 5′AMP and 5′GMP. In conjunction with adenylyl and guanylyl cyclases, PDEs are able to regulate cell signaling mechanisms that are mediated by cAMP and cGMP by reducing available intracellular pools. These second messengers play a critical role in the transduction of extracellular signals to intracellular compartments.

When a suitable agonist binds to the cell surface, adenylyl cyclase activates and turns Mg+²-ATP into cAMP. cAMP modulates the activity of the majority, if not of all, of the cells contributing to the pathophysiology of various respiratory and cardiovascular disorders. Overall, higher levels of intracellular cAMP result in improved smooth muscle constriction symptoms, while a decrease in levels of cAMP in inflammatory cells triggers the release of the inflammatory cellular mediators mentioned previously, resulting in the symptoms characteristic of asthma (e.g., bronchial smooth muscle constriction). See e.g., Giembycz, M., et al., Drugs 59:193-212 (2000).

It follows that an increase of cAMP concentration yields beneficial effects such as airway and vascular smooth muscle relaxation, inhibition of the mast cell mediator release (basophil granulose cells), suppression of the neutrophil and basophil degranulation, and inhibition of the monocyte and macrophage activation. Thus, compounds capable of inhibiting cAMP-specific PDEs could suppress the undesired activation of the bronchial smooth muscle and of a great number of inflammatory cells.

To date, eleven distinct isoenzymes (subtypes) of PDEs have been identified according to molecular structure, each with unique catalytic properties, substrate specificities, and tissue expression patterns. See Uckert, S., et al., World J Urol, 19:14-22 (2001). Non-specific PDE inhibitory compounds are able to block the activity of more than one PDE isoenzyme, often resulting in adverse side effects. Some of the adverse effects associated with, for example, the non-selective PDE inhibitor theophylline, include hypotension, tachycardia, headache, and emesis.

However, during the course of the discovery of these different families, selective inhibitors with selectivity for specific PDE isoenzymes have been designed and synthesized. PDE subtype 3 (PDE-3) and PDE subtype 4 (PDE-4) have been identified as having specific catalytic activity for cAMP, therefore inhibitory compounds targeting these particular isoenzymes result in higher levels of cAMP in the cell.

PDE subtype 4 (PDE-4) is distinguished by various kinetic properties including low Michaelis constant for cAMP and sensitivity to certain drugs. The PDE-4 enzyme family consists of four genes, which produce 4 isoforms of the PDE-4 enzyme designated PDE-4A, PDE-4B, PDE-4C, and PDE-4D. See Wang, et al., Biochem. Biophys. Res. Comm., 234:320-324 (1997). In addition, various splice variants of each PDE-4 isoform have been identified.

Of particular importance is that selective inhibition of PDE-4 activity has been found to increase intracellular levels of cAMP, leading to smooth muscle relaxtion. See Dal Piaz, V. and Giovannoni, M. P., Eur. J. Med. Chem., 35:463-480 (2000). As a result, clinicians are actively searching for compounds that inhibit PDE-4 activity and thus, have efficacy against the processes of asthma and hypertension. See e.g., Barnette, M., Progress in Drug Research (Jucker, E., ed.), vol. 53, Birkhauser Verlag, Vasel, Switzerland (1999). In fact, several PDE-4 inhibitors have been reported to improve the airflow obstruction seen in asthmatic patients by reversing or preventing bronchoconstriction, limiting airway edema (microvascular leakage), altering mucus secretion and clearance, relaxing airway smooth muscle, reducing secretion of pro-inflammatory mediators, blocking leukocyte adhesion to vascular endothelial cells, and blocking generation of oxygen-derived free radicals.

Although there are beneficial consequences to treatment with PDE-4 inhibitors, many of these agents are associated with unwanted central nervous system and gastrointestinal side effects. See Martin, C., et al., Naunyn Schmiedebergs Arch. Pharmacol., 365:284-289 (2002). Clinical use of the “first-generation” PDE-4 inhibitor rolipram, initially developed as an antidepressant, was limited by associated increase in gastric acid secretion, nausea, and vomiting. See Torphy, T. J. and Undem, B. J., Thorax, 46:512-523 (1991). Improved “second-generation” PDE4 inhibitors, such as cilomilast, lirimilast, and roflumilast, have been developed, and seem to maintain high anti-inflammatory activity while partially overcoming these side effects. See Barnette, M. S., et al., J. Pharmacol. Exp. Ther., 284:420-426 (1998).

Currently, cAMP-specific PDE inhibitors are under rigorous research and development by many pharmaceutical companies. Although some cAMP-specific PDE inhibitors have been FDA approved for clinical use, such as milrinone for heart failure and rolipram for central nervous system depression, the clinically available PDE inhibitors are ineffective for asthmatic and hypertensive conditions due to their broad spectrum of PDE inhibition, which causes many side effects at therapeutic dose.

Finally, several 2-aminopyrrole analogs of lidocaine were reported in the 1979 publication of Johnson R W, Keenan T H, Kosh J W and Sowell J W, Synthesis of Substituted 2-Aminopyrrole Analogs of Lidocaine II, Journal of Pharmaceutical Sciences 68:955-8 (1979). These analogs were synthesized while searching for better local anesthetic and antiarrhythemic agents. The compounds were diethyl substituted at the tertiary amine and hydrido substituted at the methyl group preceding the tertiary amine. Although, the benzyl carbamyl analog, one of the eight substituted 2-diethylaminoacetamido-3-carbamyl-4-methylpyrroles was reported to be able to decrease certain heart arrythemias, the therapeutic use of the benzyl carbamyl substitute was ruled out due to its low water solubility and disproportionate precipitation in blood stream. In fact, the troublesome precipitation of the benzyl compound led the research and development on these analogs being completely halted for about 20 years. The publication failed to disclose methods for preventing and/or treating hypertension and asthma. The publication also failed to disclose the phosphodiesterase enzyme as a possible site of action for these compounds.

Although there are many conventional therapeutic drugs available for the treatment of cardiovascular and respiratory disorders, none of the therapies alone are completely effective for overcoming the varied and complex symptoms of each, yet have substantially no side effects. Indeed, although conventional approaches for treating the disorders described above have been beneficial, the extremely high incidence of mortality associated with them indicates that improved treatments are still needed.

### SUMMARY

Briefly therefore, the present invention is directed to a novel compound having the structure of formula I: 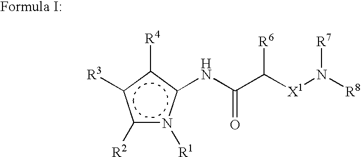
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl;
with the proviso that: when R⁶ is hydrogen, R⁷ is other than ethyl and/or X¹ is other than methyl; when R⁷ is ethyl, R⁶ is other than hydrogen and/or X¹ is other than methyl; and when X1 is methyl, R⁶ is other than hydrogen and/or R⁷ is other than ethyl; and
including the isomers, racemates, salts, and prodrugs thereof.

The present invention is also directed to a novel therapeutic composition comprising a compound having the structure described in formula I.

The present invention is also directed to a novel pharmaceutical composition comprising at least one compound compound having the structure described in formula I and a pharmaceutically acceptable excipient.

The present invention is also directed to a novel method of preventing or treating a cardiovascular or respiratory disorder in a subject, the method comprising administering to the subject an effective amount of a compound having the structure: 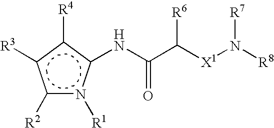
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl; and
including the isomers, racemates, salts, and prodrugs thereof.

The present invention is also directed to a novel method of preventing or treating a cardiovascular or respiratory disorder in a subject, the method comprising administering to the subject a PDE inhibitor in combination with a calcium channel blocker, wherein the PDE inhibitor and the calcium channel blocker are the same compound.

A method of modulating the activity of PDE in a subject in need of such modulation, the method comprising administering to the subject a compound having the structure: 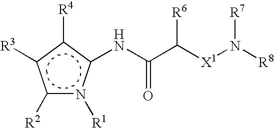
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl; and
including the isomers, racemates, salts, and prodrugs thereof.

A method of modulating the activity of L-type calcium channels in a subject in need of such modulation, the method comprising administering to the subject a compound having the structure: 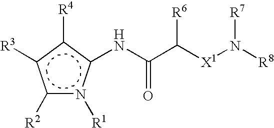
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl; and
including the isomers, racemates, salts, and prodrugs thereof.

A method of modulating the activity of PDE and L-type calcium channels in a subject in need of such modulation, the method comprising administering to the subject a compound having the structure: 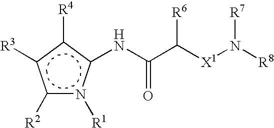
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl; and
including the isomers, racemates, salts, and prodrugs thereof.

The present invention is also directed to a novel method of preventing or treating a respiratory disorder in a subject, the method comprising administering to the subject a β-adrenergic agonist compound in combination with a compound having the structure: 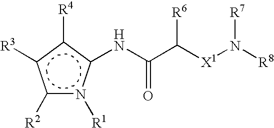
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl; and
including the isomers, racemates, salts, and prodrugs thereof.

Among the several advantages found to be achieved by the present invention, therefore, may be noted the provision of novel compositions and methods for the prevention or treatment of cardiovascular or respiratory disorders, and in particular, for the prevention or treatment of hypertension or asthma, and the provision of such compositions and methods that are efficacious, safe, and easy to administer.

### BRIEF DESCRIPTION OF THE DRAWINGS

FIG. 1 shows the typical vascular relaxation responses produced by MS23 in both arterial (panel a) and vein (panel b) tissues;

FIG. 2 shows the typical bronchial relaxation responses produced by MS23;

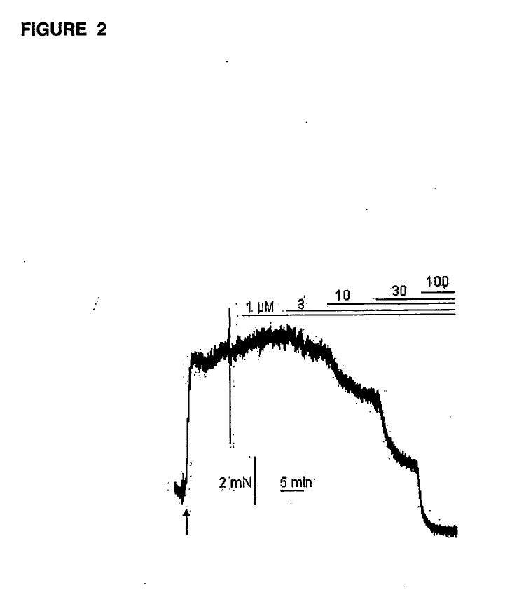

FIG. 3 shows the effects of MS23 on rat carotid blood pressure and heart rate when administered intravenously (bolus injection, Panel a and b). Lidocaine's effects were included for comparison (Panel c);

FIG. 4 shows that MS23 relaxes high K⁺ caused-contraction of porcine kidney artery rings;

FIG. 5 shows a tension assay trace of the blood vessel ring relaxation action of MS23;

FIG. 6 shows the inhibition of phosphodiesterase activity in guinea pig brain extract by MS23 measured as the ratio of conversion of cAMP to 5′-AMP by using 3H-cAMP radioisotope;

FIG. 7 shows a non-invasive blood pressure measurement using the tail-cuff method in awake SHR rats after administering saline (A) and MS23 (B) via oral gavage;

FIG. 8 shows the average data of non-invasive blood pressure measurement in awake SHR rats after administering saline or MS23 (20 mg/Kg) via oral gavage;

FIG. 9 shows the effect of MS23 on ventricular action potentials;

FIG. 10 shows the average data depicting APD shortening by various concentrations of MS23; and

FIG. 11 shows voltage-clamp data that indicates that MS23 can directly inhibit L-type calcium channels.

### DETAILED DESCRIPTION OF THE PREFERRED EMBODIMENTS

In accordance with the present invention, it has been discovered that certain compounds described herein can inhibit the activity of the phosphodiesterase enzyme (PDE), and in particular, the PDE subtype 4 (PDE-4). Many of these compounds exhibit their inhibitory effect at low concentrations—having in vitro PDE-4 inhibition IC50 values of about 2.0 μM. Accordingly, these compounds can be potent and effective drugs for use in the inhibition of the PDE, and of special value in subjects where such inhibition would be useful. For example, they can be used for the prevention or treatment of cardiovascular and/or respiratory disorders.

As used herein, the terms “PDE inhibitor” or “PDE inhibitory compounds” include any compound that inhibits, disrupts or degrades the activity of the phosphodiesterase enzyme by interfering with the phosphodiesterase enzyme's association with its substrate or by interfering with the synthesis of the PDE protein itself. In one embodiment, the compound inhibits PDE through direct contact. In specific embodiments, the contact is at a singular point. In other embodiments, the contact is through multiple and distinct contacts with residues in the protein.

The present PDE inhibitory compounds inhibit the activity of the PDE enzyme. As a group, these compounds may be referred to herein as “PDE inhibitors”, or “PDE inhibiting compounds” or “PDE inhibiting agents”. When it is said that a subject compound inhibits PDE, it is meant that the PDE enzymatic activity is lower in the presence of the compound than it is under the same conditions in the absence of such compound. One method of expressing the potency of a compound as a PDE inhibitor is to measure the “IC50” value of the compound. The IC50 value of a PDE inhibitor is the concentration of the compound that is required to decrease the PDE enzymatic activity by one-half. Accordingly, a compound having a lower IC50 value is considered to be a more potent inhibitor than a compound having a higher IC₅₀ value.

Compounds that have a high degree of PDE inhibiting activity offer advantages in therapeutic uses, because therapeutic benefits can be obtained by the administration of lower amounts of the present compounds than with less active compounds. Such highly active compounds also result in fewer side effects and in some embodiments, demonstrate a selectivity for cAMP-specific PDEs over the inhibition of cGMP-specific PDEs, and in other embodiments, demonstrate a selectivity for PDE-4 inhibition over the inhibition of other PDE isoenzyme subtypes.

In practice, the selectivity of a PDE-4 inhibitor varies depending upon the condition under which the test is performed and on the inhibitors being tested. However, for the purposes of this specification, the selectivity of a PDE-4 inhibitor can be measured as a ratio of the in vitro or in vivo IC50 value for inhibition of any other isoform of the phosphodiesterase enzyme (X) other than PDE-4, divided by the “IC₅₀ value for inhibition of PDE-4 (PDEX IC₅₀/PDE-4 IC₅₀), where X identifies any PDE other than PDE-4. As used herein, the term “IC50” refers to the concentration of a compound that is required to produce 50% inhibition of PDE activity.

A PDE-4 selective inhibitor is any inhibitor for which the ratio of PDEX IC₅₀ to PDE-4 IC50 is greater than 1. In preferred embodiments, this ratio is greater than 2, more preferably greater than 10, yet more preferably greater than 100 and more preferably still greater than 1000. Such preferred selectivity may indicate an ability to reduce the incidence of side effects incident to the administration of a PDE inhibitor to a subject. Therefore, in some embodiments, one or more of the compounds described herein is a selective PDE-4 inhibitor.

To determine whether a compound is a selective PDE-4 inhibitor, the putative inhibitor compound is typically incubated together with each individual isoform of PDE and simultaneously with substrate cyclic nucleotides. PDE inhibition is then determined by the presence or absence of substrate degradation products. See e.g. Thompson, W., et al., Adv. Cyclic Nucleotide Res., 10: 69-92 (1979); Hatzelmann, A., et al., J. Pharm. Exper. Therap., 291(1):267-279 (2001); and U.S. Pat. No. 5,712,298 to Amschler. The relative ability of an inhibitory compound to slow or prevent the degradation of tritiated cyclic nucleotides is one test that is indicative of how well the compound in question selects one or more of each isoform to inhibit. See Hatzelmann, A., et al., J. Pharm. Exper. Therap., 291(1):267-279 (2001). Representative PDE isoform enzymes and other reaction substrates can be obtained by isolation from appropriate tissues and their purchase has been reported. Id.

Thus, preferred PDE-4 selective inhibitors of the present invention have a PDE-4 IC₅₀ of less than about 50 μM, more preferred of less than about 10 μM, even more preferred of less than about 1 μM, and more preferred still of less than about 0.1 μM. Preferred PDE-4 selective inhibitors have a PDEX IC₅₀ of greater than about 50 μM, and more preferably of greater than 100 μM.

By way of example, in Hatzelmann, A., et al., J. Pharm. Exper. Therap., 291(1):267-279 (2001), the IC₅₀ for roflumilast activity on PDE-4 was reported to be 0.0008 μM, while the IC₅₀ for roflumilast activity on PDE1 was reported to be >10 μM. Accordingly, the selectivity of roflumilast for PDE-4 as compared with PDE1 would be >10/0.0008 or at least about 12,500. Likewise, the selectivity of roflumilast for PDE-4 as compared with PDE5 would be 8/0.0008 or at least about 10,000.

The present invention provides novel compounds having the structure shown in formula I: 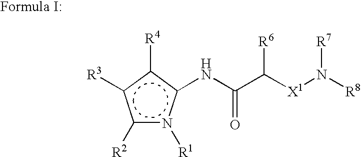
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl;
with the proviso that: when R⁶ is hydrogen, R⁷ is other than ethyl and/or X¹ is other than methyl; when R⁷ is ethyl, R⁶ is other than hydrogen and/or X¹ is other than methyl; and when X¹ is methyl, R⁶ is other than hydrogen and/or R⁷ is other than ethyl; and
including the isomers, racemates, salts, and prodrugs thereof.

The meaning of any substituent at any one occurrence in any general chemical formula herein is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, more preferably from 2 to about 4 carbon atoms, even more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as: 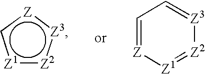
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 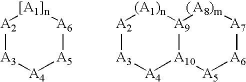
where:
when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m is greater than or equal to 0, atoms separated by 2 atoms (i.e., A₁ and A₄) are sp3 O, S, NR^{x}, CR^{x}R^{y}, and remaining A₁-A₈ are independently CR^{x} or N, and A₉ and A₁₀ are carbon.

In either, “heterocyclyl” or “heteroaryl”, the point of attachment to the molecule of interest can be at the heteroatom or elsewhere within the ring.

The term “cycloalkyl” means a mono- or multi-ringed carbocycle wherein each ring contains three to about ten carbon atoms, preferably three to about six carbon atoms, and more preferably three to about five carbon atoms. Examples include radicals, such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine. The term “cycloalkenyl” embraces unsaturated radicals having three to ten carbon atoms, such as cylopropenyl, cyclobutenyl, cyclopentenyl, cyclohexenyl, and cycloheptenyl.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂(—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

As used herein, the term “carbamoyl” refers to a carbonyl group covalently bonded at the oxo carbon to an amino group.

As used herein, the term “hydroxamate” refers to a carbonyl group covalently bonded at the oxo carbon to an amino group, wherein the amino group is in turn bonded to a hydroxyl group.

The term “oxime” means a radical comprising ═NOH.

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 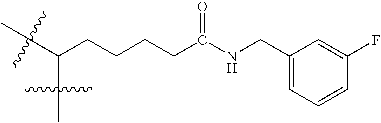
may be referred to generally as a “haloarylalkylaminocarbonylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{Y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

The present invention also provides novel compounds having the structure shown in formula I, wherein:
R¹ and R³ are independently selected from —H, C₁-C₆ alkyl, C₁-C₆ alkenyl, C₁-C₆ alkynyl, —CONR⁵R⁹, alkyl-CONR⁵R⁹, C₁-C₆ alkylthio-R¹⁰, thio-(C₁-C₆) alkyl, amino-(C₁-C₆) alkyl, C₁-C₆ alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, C₁-C₆ alkyl, C₁-C₆ alkenyl, C₁-C₆ alkynyl, —CONR⁵R⁹, alkyl-CONR⁵R⁹, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, C₁-C₆ alkyl, —CONR⁵R⁹, alkyl-CONR⁵R⁹, C₁-C₆ alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl;
with the proviso that: when R⁶ is hydrogen, R⁷ is other than ethyl and/or X¹ is other than methyl; when R⁷ is ethyl, R⁶ is other than hydrogen and/or X¹ is other than methyl; and when X¹ is methyl, R⁶ is other than hydrogen and/or R⁷ is other than ethyl; and
including the isomers, racemates, salts, and prodrugs thereof.

The present invention also provides novel compounds having the structure shown in formula I, wherein:
R¹ and R³ are independently selected from —H, C₁-C₆ alkyl, C₁-C₆ alkenyl, C₁-C₆ alkynyl, carbamyl, carbamylalkyl, C₁-C₆ alkylthio-R¹⁰, thio-(C₁-C₆) alkyl, amino-(C₁-C₆) alkyl, C₁-C₆ alkylamino-R¹⁰, cycloalkyl, aryl, and aralkyl, wherein R¹ and R³ are independently substituted or unsubstituted, which if substituted, are substituted with a halo substituent;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰, wherein R² is independently substituted or unsubstituted, which if substituted, is substituted with a halo substituent;
R⁴ is carbamyl;
R⁶ is selected from —H and C₁-C₆ alkyl;
R⁷ and R⁸ are independently C₁-C₆ alkyl;
X¹ is optionally present, and if present, is C₁-C₄ alkyl;
with the proviso that: when R⁶ is hydrogen, R⁷ is other than ethyl or X¹ is other than methyl; when R⁷ is ethyl, R⁶ is other than hydrogen or X¹ is other than methyl; and when X¹ is methyl, R⁶ is other than hydrogen or R⁷ is other than ethyl; and
including the isomers, racemates, salts, and prodrugs thereof.

The present invention also provides novel compounds having the structure shown in formula I, wherein:
R¹ and R³ are independently selected from —H, C₁-C₆ alkyl, carbamyl, carbamylalkyl, C₁-C₆ alkylthio-R¹⁰, and C₁-C₆ alkylamino-R¹⁰;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R⁴ is carbamyl;
R⁶ is selected from —H and C₁-C₄ alkyl;
R⁷ and R⁸ are independently selected from C₁-C₄ alkyl;
X¹ is absent;
with the proviso that: when R⁶ is hydrogen, R⁷ is other than ethyl; when R⁷ is ethyl, R⁶ is other than hydrogen; and
including the isomers, racemates, salts, and prodrugs thereof.

The present invention also provides novel compounds having the structure shown in formula I, wherein:
R¹ and R³ are independently selected from —H, C₁-C₆ alkyl, —CONR⁵R⁹, C₁-C₆ alkylthio-R¹⁰, and C₁-C₆ alkylamino-R¹⁰;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R⁴ is carbamyl;
R⁶ is selected from —H and C₁-C₄ alkyl;
R⁷ and R⁸ are independently selected from ethyl and propyl;
X¹ is absent;
with the proviso that: when R⁶ is hydrogen, R⁷ is other than ethyl; when R⁷ is ethyl, R⁶ is other than hydrogen; and
including the isomers, racemates, salts, and prodrugs thereof.

The present invention also provides novel compounds having the structure shown in formula I, wherein:
R¹ is —H;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R³ is methyl;
R⁴ is carbamyl;
R⁶ is selected from —H and methyl;
R⁷ and R⁸ are independently C₁-C₄ alkyl;
X¹ is absent;
with the proviso that: when R⁶ is hydrogen, R⁷ is other than ethyl; when R⁷ is ethyl, R⁶ is other than hydrogen; and
including the isomers, racemates, salts, and prodrugs thereof.

The present invention also provides novel compounds having the structure shown in formula I, wherein:
R¹ is —H;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R³ is methyl;
R⁴ is carbamyl;
R⁶ is methyl;
R⁷ and R⁸ are independently C₁-C₄ alkyl;
X¹ is absent; and
including the isomers, racemates, salts, and prodrugs thereof.

The present invention also provides novel compounds having the structure shown in formula I, wherein:
R¹ is —H;
R² is selected from —H, C₁-C₄ alkyl, carbamyl, C₁-C₄ alkylamino-R¹⁰, C₁-C₄ alkylthio-R¹⁰;
R³ is methyl;
R⁴ is carbamyl;
R⁶ is methyl;
R⁷ and R⁸ are independently selected from ethyl and propyl;
X¹ is absent; and
including the isomers, racemates, salts, and prodrugs thereof.

The present invention also provides novel compounds having the structure shown in formula I, wherein:
R¹ is —H;
R² is selected from —H, C₁-C₄ alkyl, carbamyl, C₁-C₄ alkylamino-R¹⁰, C₁-C₄ alkylthio-R¹⁰;
R³ and R⁶ are methyl;
R⁴ is carbamyl;
R⁷ and R⁸ are independently selected from ethyl and propyl;
X¹ is absent; and
including the isomers, racemates, salts, and prodrugs thereof.

The present invention also provides novel compounds having the structure shown in formula I, wherein:
R¹ is —H;
R² is selected from —H, methyl, ethyl, carbamyl, dimethylthio, methylthioethyl, ethylthiomethyl, diethylthio, dimethylamino, and methylaminoethyl;
R³ and R⁶ are methyl;
R⁴ is carbamyl;
R⁷ and R⁸ are independently selected from ethyl and propyl;
X¹ is absent; and
including the isomers, racemates, salts, and prodrugs thereof.

The present invention also provides novel compounds having the structure shown in formula I, wherein:
R¹ is —H;
R² is selected from —H, dimethylthio, methylthioethyl, ethylthiomethyl, and diethylthio;
R³ and R⁶ are methyl;
R⁴ is carbamyl;
R⁷ is propyl;
R⁸ is ethyl;
X¹ is absent; and

In one embodiment, the present invention provides a novel compound having the structure described by formula I, wherein the compound has the structure: 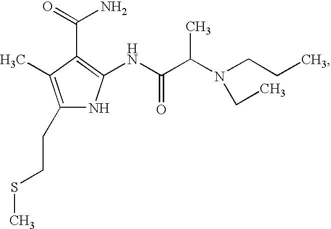
,including the isomers, racemates, salts, and prodrugs thereof. The aforementioned structure has the chemical name (2-[2-(N-ethyl-N-n-propyl) amino] propionamido-3-carbamyl-4-methyl-5-(methylthio) pyrrole), hereinafter referred to as (“MS23”).

The present group of compounds described by formula I have been discovered to be effective for the prevention and/or treatment of cardiovascular conditions (including systemic hypertension, angina pectoris, hypertension-related stroke and heart failure) and respiratory disorders such as spasmodic asthma and chronic obstructive pulmonary disease (COPD). Experimental data indicates that MS23 relaxes smooth muscles, increases coronary flow rate, reduces blood pressure, and slows the heart rate.

The responses elicited by the compound described by formula I are faster and stronger than that induced by conventional treatment agents, such as β-adrenergic agonists or calcium channel blockers, but slower and weaker than that caused by nitroglycerin. At least one of these compounds reduces heart rate and myocardium contraction markedly less than β-adrenergic agonists and calcium channel blockers. Therefore, such compounds could be a better choice in treatment of hypertensive condition, hypertension-related stroke and heart failure. The ability of relaxation of bronchial smooth muscles indicates that the compounds can be used to treat patients with spasmodic asthma, especially, in asthma patients who also have high blood pressure. The effectiveness when applied intravenously and orally ensures the feasibility of using the compounds to treat either hypertensive crises or sustained hypertensive conditions.

The present compounds may also be effective as an adjunct therapy to enhance the antiasthmatic effect of the β-adrenergic agonists, by reducing their dose, and therefore, their unwanted side effects.

These compounds are soluble in acidic solution (˜30 mg/ml, pH=3), light and heat stable, bioavailable via oral gavage, with a reasonable partition coefficient (i.e., with a calculated Logp value of 1.79), relatively low toxicity, reliable efficacy, and acceptable potency.

In vitro testing of the compounds has shown that they can markedly relax high-potassium-induced contraction (static muscle shortening) of artery, vein, and bronchial smooth muscles. In whole animal studies of Sprague-Dawley rats, the compound MS23 reduces the blood pressure when it was administered intravenously or via oral gavage. The relaxation effects on smooth muscle and the decrease of blood pressure are concentration-dependant and reversible upon removal of the compound.

At least one of the present compounds has several distinct advantages of conventional treatment agents. For example, MS23 has a fast onset of therapeutic effect (i.e., response occurs within 30 minutes after oral gavage, <3 minutes after intraperatoneally, immediate after intravenously), has less adverse effects (not affecting heart rate and myocardium contraction; does not cause baroreflex responses), low toxicity, no desensitization, it's effects are reversible and repeatable, relatively selective in action on airway and vascular smooth muscles, similar potency and efficacy in arteries and veins, soluble in physiological saline, targets a signaling-transduction mechanism that is important for smooth muscle relaxation, and has a high yield and could be a low cost therapy for cardiovascular and respiratory disorders.

In one embodiment of the present invention, the aforementioned MS23 compound has a measured IC₅₀ value of about 2 μM for inhibition of PDE-4 and a measured IC₅₀ value of about 60 μM for inhibition of PDE-3. As is apparent from the measured IC₅₀ data, MS23 is a strong inhibitor of PDE-4. Thus, the MS23 compound, in one embodiment, is a cAMP-specific phosphodiesterase inhibitor, and in particular, is an inhibitor of both cAMP-specific phosphodiesterase-3 and cAMP-specific phosphodiesterase-4. In some embodiments, MS23 is not an inhibitor of any other subtypes (e.g., other than PDE-3 and PDE-4) of the phosphodiesterase enzyme. Thus, in one embodiment, MS23 is a selective PDE inhibitor, and in particular, is a selective PDE-3/PDE-4 inhibitor.

In addition, several of the compounds described herein have the unique ability to inhibit both PDE and L-type calcium channels. Thus, it has been discovered that in one embodiment of the present invention, that certain PDE inhibitor compounds can act both as a PDE inhibitor and as a calcium channel blocker (CCB) as well. The present invention encompasses such dual-action compounds. These dual action compounds are referred to herein as “dual PDE/Ca²⁺-channel inhibitors” or “dual PDE/Ca²⁺-channel inhibiting compounds”. Thus, in one embodiment, the present invention encompasses compounds that act both as a PDE inhibitor and as a CCB.

As used herein, the phrase “calcium channel blocker” or “CCB” is intended to embrace one or more compounds or agents having the ability to interact with and block calcium transport through calcium channels located on various body tissues which are associated with mediating one or more biological functions or events such as smooth muscle or cardiac muscle contraction. In one embodiment, the compound inhibits calcium channels through direct contact with at least one calcium channel protein. In specific embodiments, the contact is at a singular point. In other embodiments, the contact is through multiple and distinct contacts with residues in the protein.

Therefore, the present invention also provides a novel method of preventing or treating a cardiovascular or respiratory disorder in a subject, the method comprising administering to the subject a PDE inhibitor in combination with a calcium channel blocker, wherein the PDE inhibitor and the calcium channel blocker are the same compound (e.g., a dual PDE/Ca²⁺-channel inhibitor). In some embodiments, the dual PDE/Ca²⁺-channel inhibitor comprises one or more of the compounds described by formula I herein. For example, in some embodiments, the compound designated MS23 is a dual PDE/Ca²⁺-channel inhibitor. Accordingly, the present invention also provides a method of modulating the activity of both PDE and calcium channels in a subject in need of such modulation, the method comprising administering to the subject a compound having the structure described in formula I. In other embodiments, the aforementioned dual PDE/Ca²⁺-channel inhibiting compounds act both as a cAMP-specific PDE inhibitor and as an L-type CCB. In still other embodiments, the aforementioned dual PDE/Ca²⁺-channel inhibiting compounds act both as a PDE-4 inhibitor and as an L-type CCB.

The methods and compositions of the present invention would be useful, for example, to reduce such cardiovascular disorder symptoms as hypertension in a subject suffering from such symptoms. The methods and compositions of the present invention would also be useful to prevent the occurrence of such symptoms. Likewise, the methods and compositions of the present invention would also be useful, for example, to reduce such respiratory disorder symptoms as coughing and shortness of breath in a subject suffering from such symptoms.

The methods and compositions of the present invention would also be useful for the treatment of cardiovascular disorder-related complications or respiratory disorder-related complications, which may arise indirectly from having a cardiovascular or respiratory disorder, by treating the underlying disorder itself or a symptom thereof. For example, if a subject is suffering from a cardiovascular disorder-related complication, such as a heart failure, the treatment of the underlying disorder symptom, such as hypertension, by the methods and compositions of the present invention will likewise improve the symptoms of the associated complication.

The methods and compositions of the present invention are also useful to reduce the number of hospitalizations of subjects suffering from cardiovascular or respiratory disorders, or to prevent or retard, in subjects, the development of complications associated with cardiovascular or respiratory disorders, such as, for example, heart failure, which may eventually arise from having a such disorders.

The administration of the compounds described by formula I herein for the prevention or treatment of cardiovascular and/or respiratory disorders is an unexpectedly effective treatment and preventative monotherapy. Such administration is effective for improving the symptoms of cardiovascular and respiratory disorders and related complications while avoiding or reducing certain disadvantages of conventional treatment agents.

As used herein, the term “monotherapies” or monotherapy” is intended to embrace administration of one or more of the compounds described by formula I herein to a subject suffering from a cardiovascular or respiratory disorder or a related complication as a single therapeutic treatment without an additional conventional treatment agent.

As used herein, the terms “conventional treatment agent” or “conventional treatment agents” refer to any compound that is other than a compound described according to formula I and that has efficacy or can be later shown to have efficacy for the treatment and/or prevention of cardiovascular and/or respiratory disorders.

For example, conventional treatment agents that are β-adrenergic agonists, include, but are not limited to, metaproterenol, pirbuterol, albuterol, levalbuterol, formoterol, salmeterol, terbutaline, isoetharine, levalbuterol, salbutamol, bambuterol, fenoterol, reproterol, tulobuterol, and the like.

Accordingly, in one embodiment, the present invention is also directed to a novel method of preventing or treating a respiratory disorder in a subject, the method comprising administering to the subject one or more compounds having the structure described in formula I in combination with a β-adrenergic agonist, and in some embodiments, a β₂-adrenergic agonist.

The novel compounds described by formula I herein are useful not only for improving cardiovascular and respiratory disorder symptoms and shortening recovery times, but also for reducing the dosages of conventional treatment agents that are normally required. Reduced dosages of conventional treatment agents are beneficial where normal dosages exhibit harmful side effects or require burdensome treatment regimens. The administration of low dosages of conventional treatment agents can, in one embodiment, provide a reduction in side effects corresponding to such agents.

As used herein, the terms “lowered dosages”, “low dose”, or “low dose amount”, in characterizing a therapeutically effective amount of a compound described by formula I herein defines a quantity of such agent, or a range of quantity of such agent, that is capable of preventing or treating the symptoms of a cardiovascular or respiratory disorder or a related complication while optionally reducing or avoiding one or more side effects of a monotherapy with a conventional treatment agent.

The combination therapy of a compound described by formula I herein and conventional treatment agent may also be useful for decreasing the required number of separate dosages, thus, potentially improving patient compliance.

For example, the administration of one or more of the compounds described by formula I herein in combination with a conventional treatment agent such as a β-adrenergic agonist is an effective treatment for respiratory disorders and respiratory disorder-related complications, and in preferred embodiments, is superior to the use of either agent alone. Moreover, in preferred embodiments, the combination therapies of the present invention demonstrate a synergistic efficacy for treating and preventing respiratory disorders and respiratory disorder-related complications that is greater than what would be expected from simply combining any of the individual monotherapies. As used herein, the term “synergistic” refers to the combination of one or more of the compounds described by formula I herein and a β-adrenergic agonist as a combined therapy having an efficacy for the prevention and treatment of respiratory disorders that is greater than what would be expected merely from the sum of their individual effects. The synergistic effects of the embodiments of the present invention's combination therapies encompass additional unexpected advantages for the treatment and prevention of respiratory disorders. Such additional advantages include, but are not limited to, lowering the required dose of conventional treatment agents (e.g., β₂-adrenergic agonists), reducing the side effects of such conventional treatment agents, and rendering those agents more tolerable to subjects in need of vascular disorder therapy.

As used herein, the terms “combination therapy”, “co-administration”, “co-administering”, “administration with”, “administering”, “combination”, or “co-therapy”, when referring to the use of one or more of the compounds described by formula I herein in combination with a conventional treatment agent, such as a β-adrenergic agonist, are intended to embrace administration of each agent in a sequential manner in a regimen that will provide beneficial effects of the drug combination, and is intended as well to embrace co-administration of these agents in a substantially simultaneous manner.

Substantially simultaneous administration can be accomplished, for example, by administering to the subject one or more of the compounds described by formula I herein in combination with a conventional treatment agent, together in one therapeutic dosage form, such as in a single capsule, tablet, or injection, or in multiple separate therapeutic dosage forms, such as in separate capsules, tablets, or injections.

Sequential administration of such treatments encompasses both relatively short and relatively long periods between the administration of each of the compounds of the present method. However, for purposes of the present invention, the compound described by formula I herein is administered while the conventional treatment agent is still having an efficacious effect on the subject.

Preferably, the compound described by formula I herein is to be given to the subject within the therapeutic response time of the administered conventional treatment agent. As used herein, the terms “therapeutic response time” mean the duration of time after administration that a compound has a therapeutic effect within a subject's body.

As used herein, the terms “therapeutically effective” are intended to qualify the amount of an agent for use in a therapy that will achieve the goal of preventing or treating by improvement in the severity of the cardiovascular or respiratory disorder symptoms or related complication symptoms in a subject, while avoiding adverse side effects typically associated with conventional treatment agents.

In one embodiment, the present invention encompasses a method for preventing cardiovascular or respiratory disorders or related complications in a subject, and in preferred embodiments, preventing cardiovascular or respiratory disorders or related complications in subject that is predisposed to cardiovascular or respiratory disorders or related complications, the method comprising administering to the subject one or more of the compounds described by formula I herein alone or in combination with a conventional treatment agent.

As used herein, the terms “to prevent”, “preventing”, or “prevention” refer to any reduction, no matter how slight, of a subject's predisposition or risk for developing a cardiovascular or respiratory disorder or related complication. For purposes of prevention, the subject is any subject, and preferably is a subject that is at risk for, or is predisposed to, developing a cardiovascular or respiratory disorder or related complication. As used herein, the terms “predisposition, “predisposed to” or “at risk for,” all of which may be used interchangeably herein, includes any subject with an increased chance for developing a cardiovascular or respiratory disorder or related complication. The subject may be at risk due to genetic predisposition, environment, trauma, sex, age, lifestyle, diet, exposure to cardiovascular or respiratory disorder causing agents and/or having physiological factors such as anatomical and biochemical abnormalities, and the like. The subject may also be at risk for re-developing a cardiovascular or respiratory disorder or related complication after suffering from a cardiovascular or respiratory disorder or related complication.

In another embodiment, the present invention encompasses a method for treating cardiovascular or respiratory disorders or related complications in a subject, and in preferred embodiments, treating cardiovascular or respiratory disorders or related complications in subject that is suffering from a cardiovascular or respiratory disorder or related complication, the method comprising administering to the subject one or more of the compounds described by formula I herein alone or in combination with a conventional treatment agent.

As used herein, the terms “treating”, “treatment”, “treated”, or “to treat,” mean to alleviate symptoms, eliminate the causation either on a temporary or permanent basis, or to alter or slow the appearance of symptoms or symptom worsening.

In yet another embodiment, the present invention provides a method of modulating the activity of PDE in a subject in need of such modulation, the method comprising administering to the subject a compound having the structure described in formula I. In other embodiments the present invention provides a method of modulating the activity of L-type calcium channels in a subject in need of such modulation, the method comprising administering to the subject a compound having the structure described in formula I.

In accordance with the present invention, any composition comprising one or more of the compounds described by formula I alone or in combination with a conventional treatment agent may be administered to a subject according to standard routes of drug delivery that are well known to one of ordinary skill in the art.

The compounds can be supplied in the form of a salt, a prodrug, an isomer, a tautomer, a racemic mixture, or In any other chemical form or combination that, under physiological conditions, still provides for inhibition of PDE. The present invention includes all possible diastereomers as well as their racemic and resolved, enantiomerically pure forms.

The compounds useful in the present invention can have no asymmetric carbon atoms, or, alternatively, the useful compounds can have one or more asymmetric carbon atoms. When the useful compounds have one or more asymmetric carbon atoms, they, therefore, include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention. Also included in the methods, combinations and compositions of the present invention are the tautomeric forms of the described compounds.

Also included in the methods and compositions of the present invention are the prodrugs of the described compounds and the pharmaceutically-acceptable salts thereof.

As used herein, the term “prodrug” refers to drug precursor compounds which, following administration to a subject and subsequent absorption, are converted to an active species in vivo via some process, such as a metabolic process.

The compounds described herein can be of any purity or grade, as long as the preparation is of a quality suitable for pharmaceutical use. The compounds of formula I can be provided in pure form, or they can be accompanied with impurities or commonly associated compounds that do not affect their physiological activity or safety.

The term “pharmaceutically acceptable” is used adjectivally herein to mean that the modified noun is appropriate for use in a pharmaceutical product.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Exemplary pharmaceutically acceptable salts include the salts of hydrochloric acid and trifluoroacetic acid.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p.1418, the disclosure of which is hereby incorporated by reference only with regards to the disclosures of pharmaceutically acceptable salts.

In another embodiment of the present invention, the compounds of formula I can be provided in a “pharmaceutically acceptable carrier” or “pharmaceutically acceptable excipient”, both of which are used interchangeably herein, to form a pharmaceutical composition.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and excipients are chosen such that side effects from the pharmaceutical compound are minimized and the performance of the compound is not canceled or inhibited to such an extent that treatment is ineffective.

The pharmaceutically acceptable carrier can also be selected on the basis of the desired route of administration of the compound. For example, in a preferred embodiment the carrier is suitable for oral administration. In some embodiments, the composition includes a carrier or additional agent that is suitable for promoting delivery of the compound to the brain. Examples of such carriers include those disclosed in U.S. Pat. Nos. 5,604,198; 5,827,819; 5,919,815; 5,955,459; and 5,977,174.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

The pharmaceutical compositions of the invention can be prepared by any of the well-known techniques of pharmacy, for example, by admixing the components.

The compounds of formula I can be administered to a subject by any conventional means and route available for use in conjunction with pharmaceuticals, either as individual therapeutic compounds or as a combination of therapeutic compounds or as independent multiple pharmaceutical compositions.

In the combination therapies, administration of two or more of the therapeutic agents useful in the methods and compositions of the present invention may take place sequentially in separate formulations, or may be accomplished by simultaneous administration in a single formulation or in a separate formulation. The formulation may be in the form of a bolus, or in the form of aqueous or non-aqueous isotonic sterile injection solutions or suspensions. For example, the therapeutic compounds of the present invention may be a combined dosage form or in separate dosage forms intended for substantially simultaneous oral administration. The therapeutic compounds may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from, for example, a few minutes to several hours to days depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval.

The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered enterally or parenterally, separately or together, each therapeutic compound may be contained in a suitable pharmaceutical formulation of any of the pharmaceutically-acceptable excipients, diluents or other formulations components described herein. Thus, the combination of therapeutic compounds may be administered by any combination of, for example, oral/oral, oral/parenteral, or parenteral/parenteral route.

Pharmaceutical compositions according to the present invention include those suitable for enteral (e.g., oral and buccal), inhalation spray, rectal, topical, or parenteral (e.g., subcutaneous, intramuscular, intravenous, intrathecal, intramammary, intramedullary and intradermal injections, or infusion techniques) administration, although the most suitable route in any given case will depend on the nature and severity of the condition being treated and on the nature of the particular compound which is being used.

In most cases, the preferred route of administration is enteral (e.g., orally). Oral administration includes solution, tablets, sustained release capsules, enteric-coated capsules, and syrups. The pharmaceutical composition may be administered in dosage unit formulations containing conventional nontoxic pharmaceutically acceptable carriers, adjuvants, and vehicles as desired. When administered, the pharmaceutical composition may be at or near body temperature.

The compounds of the present invention can be delivered orally either in a solid, in a semi-solid, or in a liquid form. Pharmaceutically acceptable carriers can be in solid dosage forms for the methods of the present invention, which include tablets, capsules, gelcaps, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Pharmaceutical compositions suitable for oral administration can be presented in discrete units each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing the compounds described herein may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents, which may be naturally-occurring phosphatides, for example lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, or antioxidants such as ascorbic acid; one or more coloring agents; one or more flavoring agents; and/or one or more sweetening agents, such as sucrose or saccharin. Solutions and suspensions may be prepared from powders or granules having one or more pharmaceutically acceptable carriers or diluents, or a binder such as gelatin or hydroxypropylmethyl cellulose, together with one or more of a lubricant, preservative, surface active or dispersing agent.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Oral delivery of the combinations of the present invention can include formulations, as are well known in the art, -to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Also encompassed by the present invention is buccal or sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The compositions of the present invention may also be administered parenterally, for example, by either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above, or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these parenteral compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Administration of the compounds described herein can also be by inhalation, in the form of aerosols or solutions for nebulizers. Therefore, in one embodiment, the compounds are administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Administration of the compounds described herein can also be by inhalation, in the form of aerosols or solutions for nebulizers. Therefore, in one embodiment, the compounds are administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Aerosols of liquid particles comprising the active materials may be produced by any suitable means, such as inhalatory delivery systems. Nebulizers are commercially available devices, which transform solutions, or suspensions of the active ingredient into a therapeutic aerosol mist by means of acceleration of compressed gas, typically either air or oxygen, through a narrow venturi orifice or by means of ultrasonic agitation. Suitable formulations for use in nebulizers consist of the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles comprising the active materials may likewise be produced with any solid particulate medicament aerosol generator. Aerosol generators for administering solid particulate medicaments to a subject produce particles, which are respirable, as explained above, and generate a volume of aerosol containing a predetermined metered dose of a medicament at a rate suitable for human administration.

One type of solid particulate aerosol generator is an insufflator. Suitable formulations for administration by insufflation include finely comminuted powders, which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by means of air drawn through the device upon inhalation or by means of a manually operated pump. The powder employed in the insufflator either consists solely of the active ingredient or of a powder blend comprising the active materials, a suitable powder diluent, such as lactose, and an optional surfactant.

A second type of aerosol generator is a metered dose inhaler. Metered dose inhalers are pressurized aerosol dispensers, typically containing a suspension or solution formulation of the Cox-2 inhibitor and the muscarinic receptor antagonist in a liquefied propellant. During use, the metered dose inhaler discharges the formulation through a valve, adapted to deliver a metered volume, to produce a fine particle spray containing the active materials. Any propellant may be used for aerosol delivery, including both chlorofluorocarbon-containing propellants and non-chlorofluorocarbon-containing propellants.

A third type of aerosol generator is a electrohydrodynamic (EHD) aerosol generating device, which has the advantage of being adjustable to create substantially monomodal aerosols having particles more uniform in size than aerosols generated by other devices or methods. Typical EHD devices include a spray nozzle in fluid communication with a source of liquid to be aerosolized, at least one discharge electrode, a first voltage source for maintaining the spray nozzle at a negative (or positive) potential relative to the potential of the discharge electrode, and a second voltage source for maintaining the discharge electrode at a positive (or negative) potential relative to the potential of the spray nozzle. Most EHD devices create aerosols by causing a liquid to form droplets that enter a region of high electric field strength. The electric field then imparts a net electric charge to these droplets, and this net electric charge tends to remain on the surface of the droplet. The repelling force of the charge on the surface of the droplet balances against the surface tension of the liquid in the droplet, thereby causing the droplet to form a cone-like structure known as a Taylor Cone. In the tip of this cone-like structure, the electric force exerted on the surface of the droplet overcomes the surface tension of the liquid, thereby generating a stream of liquid that disperses into a many smaller droplets of roughly the same size. These smaller droplets form a mist, which constitutes the aerosol cloud that the user ultimately inhales.

Administration of the compositions of the present invention can also be rectally. Pharmaceutical compositions suitable for rectal administration are preferably presented as unit-dose suppositories. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Other methods for administration of the compounds described herein include dermal patches that release the medications directly into a subject's skin. Such patches can contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

A penetration enhancer is an agent used to increase the permeability of the skin to an active agent to increase the rate at which the drug diffuses through the skin and enters the tissues and bloodstream.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and mixtures thereof.

The compositions of the present invention can optionally be supplemented with additional agents such as, for example, viscosity enhancers, preservatives, surfactants and penetration enhancers.

Viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives can be optionally employed to prevent microbial contamination of the compositions described herein. Suitable preservatives include, but are not limited to, polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-103), cyclodextrin, or other agents known to those skilled in the art. Typically, such co-solvents are employed at a level of from 0.01% to 2% by weight.

Pharmaceutically acceptable excipients and carriers encompass all the foregoing and the like. The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins), 2000; Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The therapeutic compounds described herein may be a combined dosage form or in separate dosage forms intended for substantially simultaneous oral administration. The therapeutic compounds may also be administered sequentially, with either therapeutic compound being administered by a regimen calling for two-step ingestion. Thus, a regimen may call for sequential administration of the therapeutic compounds with spaced-apart ingestion of the separate, active agents. The time period between the multiple ingestion steps may range from, for example, a few minutes to several hours to days depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds, whether administered simultaneously, substantially simultaneously, or sequentially, may involve a regimen calling for administration of one therapeutic compound by oral route and another therapeutic compound by intravenous route.

The amount of the compounds described herein that is required to achieve the desired biological effect will, of course, depend on a number of factors such as the specific compound chosen, the use for which it is intended, the mode of administration, and the host to be treated and the clinical condition of the recipient.

For purposes of the present invention, it is preferred that the amount of the compound chosen comprises an effective amount of the compound. Still further preferred is that the amount of the co-therapy with a compound described by formula I and conventional treatment agent comprises a therapeutically effective amount of the co-therapy.

As used herein, an “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

A cardiovascular disorder symptom is considered ameliorated or improved if any benefit is achieved, no matter how slight. Likewise, a respiratory disorder symptom is considered ameliorated or improved if any benefit is achieved, no matter how slight.

A “therapeutically effective amount” is intended to qualify the amount of the compound required to-treat, prevent or inhibit a cardiovascular or respiratory disorder.

As used herein, the terms “prophylactically effective” refer to an amount of a compound that causes a decrease in the frequency of incidence of a cardiovascular or respiratory disorder. The term “prophylactic” refers to the prevention of cardiovascular or respiratory disorders, whereas the term “therapeutic” refers to the effective treatment of an existing cardiovascular or respiratory disorder.

It will be appreciated that the amount of the compound(s) required for use in the treatment or prevention of a cardiovascular or respiratory disorder will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages. For purposes of calculation of dosage amounts, the weight of a normal adult human will be assumed to be 70 kg.

When the term “about” is used herein in relation to a dosage amount of a particular compound, it is to be understood to mean an amount that is within ±0.05 mg. By way of example, “about 0.1-10 mg/day” includes all dosages within 0.05 to 10.05 mg/day.

For the purposes of calculating and expressing a dosage rate, all dosages that are expressed herein are calculated on an average amount-per-day basis irrespective of the dosage rate. For example, one 100 mg dosage of a compound described herein taken once every two days would be expressed as a dosage rate of 50 mg/day. Similarly, the dosage rate of an ingredient where 50 mg is taken twice per day would be expressed as a dosage rate of 100 mg/day.

In the subject invention, the described compounds can be used in any amount that is an effective amount. It is preferred, however, that the amount of the compound(s) described by formula I that is administered is within a range of about 0.0001 mg/day per kilogram of the subject to about 100 mg/day/kg. It is more preferred that the amount of the compound be within a range of about 0.01 mg/day/kg to about 10 mg/day/kg. An amount that is within a range of about 0.01 mg/day/kg to about 5 mg/day/kg, is even more preferred. Even more preferred still, the compound(s) described by formula I should be dosed to the subject between about 0.1 and 2.0 mg/kg per day. A total daily dose of the compound can generally be in the range of from about 0.001 to about 10,000 mg/day in single or divided doses, with preferred levels of between about 0.1 mg to about 1000 mg.

While not intended to be limiting, an example of the normally prescribed dosage for the PDE inhibitor, roflumilast, has been reported to be about 0.5 mg once daily for human rhinitis treatment. See Schmidt, M. et al., J. Allergy Clin. Immunol. 108(4):530-536 (2001). In rats, roflumilast dosing has been reported to be 0.3-3 mg/kg of body weight. See Wollin, L., et al., Altana Poster Discussion, New treatments for asthma and COPD: PDE-4 inhibitors and anti-IGE (2002), <http://www.altana.com/root/index.php?lang=de&page_id=329>. In humans, roflumilast has been reported as effective when dosed at between about 0.01 and 0.5 mg/kg of body weight for inhalation and between about 0.05 and 2 mg/kg of body weight per day for systemic therapies. See U.S. Pat. No. 5,712,298 to Amschler.

Other examples of recommended PDE inhibitor dosages include: Tibenalast (150 mg per day), Piclamilast (0.2-0.8 mg per day), Rolipram (0.5-2 mg/kg per day), Arofylline (20 mg per day), RP 73401(2 mg/kg per day), SB 207499 (2.3-5 mg/kg per day), YM-976 (0.52-7.3 mg/kg per day), NVP-ABE171 (0.1-3 mg/kg per day), CDP-840 (2 mg/kg per day), and Ariflo (20-30 mg per day). See e.g. Souness, J., et al., Immunopharmacology, 47:127-162 (2000); Teixeira, M., et al., Memorias do Instituto Oswaldo Cruz, 92(II):193-196 (1997); Trifilieff, A., et al., J. Pharmacol. Exp. Ther., 301(1):241-82002 (2002).

The frequency of dose will necessarily depend upon the half-life of the particular compound. If the compound has a short half-life (e.g., from about 2 to 10 hours) it may be necessary to give one or more doses per day. Alternatively, if the half-life is longer (e.g., from about 2 to about 15 days) it may only be necessary to give a dosage once per day, per week, or even once every 1 or 2 months.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages. It is understood, however, that specific dose levels of the therapeutic agents or therapeutic approaches of the present invention for any particular subject depends upon a variety of factors including the activity of the specific compound employed, the age, body weight, general health, sex, and diet of the subject, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for subject administration. Studies in animal models also generally may be used for guidance regarding effective dosages for treatment of cardiovascular and respiratory disorders in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art.

Those skilled in the art will also appreciate that dosages may also be determined with guidance from Goodman & Gilman's The Pharmacological Basis of Therapeutics, Ninth Edition (1996), Appendix II, pp. 1707-1711.

Dosages for the therapies provided herein may be determined and adjusted based on the efficacy demonstrated in reducing or preventing the symptoms of a cardiovascular or respiratory disorder. In addition, one of ordinary skill in the art will know how to measure and quantify the presence or absence of cardiovascular or respiratory disorder symptoms.

For example, the degree and severity of hypertension can be determined by measuring the blood (arterial) pressure in the brachial artery of the arm. Blood pressure is equal to the total cardiac output multiplied by the total peripheral resistance. The systolic pressure occurs as the heart's ventricles contract and force blood into the aorta. The diastolic pressure occurs after the aortic valve closes and the pressure falls to a minimum level before the next ventricular contraction. This minimum pressure is the diastolic pressure. Hypertension occurs when the systolic and diastolic pressures depart from a normal value.

For most adults, normal blood pressures are typically less than 140 mm Hg for the systolic pressure and less than 85 mm Hg for the diastolic pressure. Pressures that exceed either of the two measurements are considered abnormal. Stage I hypertension (mild) is where the systolic pressure is between 140-159 mm Hg and the diastolic pressure is between 90-99 mm Hg. This range progresses until stage IV hypertension (very severe) is reached where the systolic pressure is greater than 210 mm Hg or the diastolic pressure is greater than 120 mm Hg.

One example of an instrument used to measure arterial blood pressure is called a sphygmomanometer. For this procedure, a rubber inflatable cuff is place over the brachial artery and the pressure in the cuff is raised until the cuff pressure exceeds that of the blood in the artery. At this point, there is no blood flow and thus no pressure. The pressure in the cuff is then slowly released and the radial pulse then reappears. The pressure at which point the pulse reappears corresponds to the systolic pressure. Alternatively, a stethoscope can be used to listen for the (Korotkov) sounds of the pressure returning within the brachial artery (systolic) and the loss of sounds of when the pressures returns in full (diastolic).

Other procedures may be employed to determine the presence or absence of a cardiovascular disorder besides measuring the arterial blood pressure. For example, several invasive and noninvasive methods may be employed to diagnose and monitor such conditions as heart failure, congestive heart failure, myocardial infarction, myocardial fibrosis, and arteriosclerosis. See The Merck Manual, 17^{th} edition, Sec. 16, Chapter 198, Diagnostic Cardiovascular Procedures.

Examples of noninvasive techniques include plain radiography of the chest to determine heart size and shape, radionulceotide imaging, myocardial perfusion imaging to determine arterial stenosis, and magnetic resonance imaging. Id. Likewise, examples of invasive techniques include venous and arterial catheterization to accurately determine blood pressures; cardiac catheterization to determine heart anatomical information and blood flow data, angiocardiography, and angioplasty to revascularize coronary arteries. Id. Anyone of ordinary skill in the art can perform and interpret these procedures to determine the effectiveness of the methods and compositions of the present invention.

Still other techniques may be employed to determine the presence or absence of cardiovascular disorders after treatment with the methods and compositions of the present invention. The severity of heart failure is classified by a system established by the New York Heart Association (NYHA). The system is divided into four classes that are based on the degree of breathlessness to indicate a severity score. For example, Class I: the patient is breathless with more than ordinary activity, Class II: breathless with ordinary activity, Class III: breathless with minimal activity, and Class IV: breathless symptoms at rest. See Chavey, W., et al., Am. Fam. Physician 1;64(5):769-74 (2001). Thus, for example, a change from class IV to class III is indicative of an improvement in the symptoms of heart failure.

In addition, one of ordinary skill can determine the efficacy of the combinations of the present invention by monitoring the physiological levels of several biological markers. For example, changes in the levels of such markers as neurohormones including natriuretic peptides, urinary aldosterone, plasma renin, and serum potassium are indicative of how well the combinations provided in the present invention are treating or preventing a cardiovascular disorder. Natriuretic peptides are a group peptides that have diverse actions in cardiovascular, renal and endocrine homeostasis. Elevated natriuretic peptide levels in the blood are generally observed in subjects under conditions of blood volume expansion and after vascular injury such as acute myocardial infarction and remain elevated for an extended time after the infarction. See U.S. Pat. No. 6,410,524 to Perez, et al. and see Uusimaa, et al., Int. J. Cardiol. 69:5-14 (1999).

Thus, blood concentrations of natriuretic peptides can be measured before and after administration of the combination therapy of the present invention in order to correlate the efficacy of a particular dosage with a reduction in the symptoms of a cardiovascular disorder. Accordingly, dosing of therapeutic compositions for cardiovascular disorders may be determined and adjusted based on measurement of blood concentrations of natriuretic peptides.

In yet another preferred embodiment, the effectiveness of a particular dosage of a compound described by formula for treating or preventing a respiratory disorder is determined and adjusted based on the efficacy demonstrated in reducing or preventing the symptoms of any respiratory disorder. One of ordinary skill in the art will know how to measure and quantify the symptoms of a respiratory disorder. For example, the degree and severity of asthma and COPD can be determined by measuring lung expiratory flow volume and expiratory flow rates. Such a measurement is accomplished with, for example, a spirometer, flow volume loop, or pneumotach, before and after each of the treatments. Use of spirometry is a standard test for determining the efficacy of medicaments after administration to a subject suffering from a pulmonary inflammatory disorder. A device called a spirometer is used to measure how much air the lungs can hold and how well the respiratory system is able to move air into and out of the lungs.

Spirometry is a medical test that measures the physical volume of air an individual forcibly inhales or exhales into a device. The objective of spirometry is to assess ventilatory function. An estimate of flow rate, or the rate at which the volume is changing as a function of time can also be calculated with spirometery. See College of Physicians and Surgeons of Alberta, “Guidelines For Spirometry & Flow Volume Measurements” (1998). Thus, with the methods of the present invention, spirometric comparisons of pulmonary airflow before and after treatment will elucidate similarities and differences that enable one of skill to determine the effectiveness of the treatment methods.

Common parameters that spirometry measures are Forced Vital Capacity (FVC)—the maximum volume of air, measured in liters that can be forcibly and rapidly exhaled. Another parameter is Forced Expiratory Volume (FEV1)—the volume of air expelled in the first second of a forced expiration. Normal parameters for a subject not suffering from an inflammatory disorder such as asthma or COPD is: Tidal volume—5 to 7 milliliters per kilogram of body weight; Expiratory reserve volume—25% of vital capacity; Inspiratory capacity—75% of vital capacity forced expiratory volume—75% of vital capacity after 1 second, 94% after 2 seconds, and 97% after 3 seconds. Healthatoz.com, wellness, test & procedures, spirometry <http://www.healthatoz.com /atoz/TestProcedures/TPspirometry.html>.

Spirometry results are expressed as a percentage, and are considered abnormal if less than 80% of the normal predicted value. An abnormal result usually indicates the presence of some degree of obstructive lung disease such as COPD and chronic bronchitis, or restrictive lung disease such as pulmonary fibrosis or asthma.

In a preferred embodiment, the methods and compositions of the present invention encompass any amelioration or improvement in the symptoms of a cardiovascular disorder as long as any benefit is achieved, no matter how slight or even if the improvement is undetectable with current techniques. Further, the symptoms of a cardiovascular disorder are considered prevented when any symptom is avoided no matter how little prevention is seen or even if the prevention is also undetectable with current techniques.

It is preferred that the methods and compositions of the present invention are used in the treatment and/or prevention of a cardiovascular or respiratory disorder in a subject, and in preferred embodiments, the subject is in need of the treatment or prevention of a cardiovascular or respiratory disorder.

As used herein, the term “subject” includes any subject, and preferably the subject is in need of the prevention or treatment of a cardiovascular or respiratory disorder.

For purposes of prevention, the term “subject” refers to any subject, and preferably is a subject that is at risk for, or is predisposed to, developing a cardiovascular or respiratory disorder.

For purposes of treatment, the term “subject” refers to any subject, and preferably is a subject that is suffering from any symptom of a cardiovascular or respiratory disorder.

As used herein, the terms “subject is in need of the treatment or prevention of a cardiovascular or respiratory disorder” refer to any subject who is suffering from or is predisposed to any cardiovascular or respiratory disorder described herein. The terms “subject is in need of the prevention or treatment of a cardiovascular or respiratory disorder” also refers to any subject that requires a lower dose of conventional treatment agents. In addition, the term “subject is in need of the prevention or treatment of a cardiovascular or respiratory disorder,” means any subject who requires a reduction in the side-effects of a conventional treatment agent. Furthermore, the terms “subject is in need of the prevention or treatment of a cardiovascular or respiratory disorder,” means any subject who requires improved tolerability to any conventional treatment agent.

In preferred embodiments, the methods and compositions of the present invention encompass the prevention and/or treatment of one or more cardiovascular disorders chosen from myocardial ischemia, transient ischemic attack, hypertension, hypotension, heart arrhythmias, including atrial fibrillation and flutter, tachycardia, and ventricular fibrillation, pulmonary hypertension, hypokalemia, angina pectoris, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis arteriosclerosis, atherosclerosis, calcification (such as vascular calcification and valvar calcification), coronary artery disease, coronary heart disease, peripheral arterial disease, heart failure, congestive heart failure, shock, stroke, left ventricular hypertrophy, angina, diabetic nephropathy, kidney failure, eye damage, cardiac damage, diabetic cardiac myopathy, renal insufficiency, renal injury, renal arteriopathy, peripheral vascular disease, left ventricular hypertrophy, cognitive dysfunction, headache, aortic aneurysm, deep vein thrombosis, bacterial endocarditis, cardiomyopathy, congenital cardiovascular defects, rheumatic heart disease, valvular heart disease, Adams-Stokes disease, antiphospholipid syndrome, aortic regurgitation, long Q-T syndrome, Marfan syndrome, Raynaud's syndrome, Wolff-Parkinson-White syndrome (WPW).

In other preferred embodiments, the methods and compositions of the present invention encompass the prevention and/or treatment of one or more respiratory disorders chosen from asthma, spasmodic asthma, bronchitis, chronic obstructive pulmonary disease (COPD), cystic fibrosis, pulmonary embolism, pneumonia, pulmonary fibrosis, respiratory failure, acute respiratory distress syndrome, bronchiectasis, rhinitis, chronic rhinitis, sinusitis, chronic sinusitis, emphysema, pulmonary sarcoidosis, tuberculosis, alpha-1 antitrypsin deficiency, allergies, alveolar capillary dysplasia, asbestosis, black lung, bronchiolitis, cold, goodpasture syndrome, laryngeal cancer, laryngomalacia, legionnaires' disease, lung cancer, lymphagioleiomyomatosis (LAM), persistent cough, pleurisy (Pleuritis), Pneumothorax, Respiratory Syncytial Virus (RSV), severe acute respiratory syndrome (SARS), silicosis, sinus infection, tonsillitis, valley fever, recurrent respiratory papillomatosis, bronchopulmonary dysplasia (BPD), influenza, hantavirus pulmonary syndrome (HPS), hayfever, primary ciliary dyskinesia (PCD), kartagener's syndrome, lymphangioleiomyomatosis (LAM), mesothelioma, primary pulmonary hypertension (PPH), spontaneous pneumothorax, meningococcemia, and wegener's granulomatosis.

In preferred embodiments, the methods and compositions of the present invention encompass the prevention and/or treatment of hypertension and/or asthma.

The methods and compositions of the present invention not only encompass the prevention or treatment of cardiovascular or respiratory disorders in humans, but also in several animals. For example, although not as common as in humans, animals have been reported to also suffer adverse consequences related to cardiovascular and respiratory disorders. Accordingly, besides being useful for humans, the methods and compositions of the present invention also encompass the treatment and prevention of cardiovascular or respiratory disorders in any animal, including other mammals, such as horses, dogs, cats, rats, mice, sheep, pigs, cattle, hamsters, gerbils, and the like. Transgenic animals (i.e., where the cardiovascular or respiratory disorder has been artificially induced), including transgenic mammals, such as transgenic mice and rats, are also encompasses by the present invention. Thus, it is preferred that the subject is an animal, and yet more preferred, the subject is a mammal. Preferably, the mammal is a human.

The following examples describe embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples. In the examples, all percentages are given on a weight basis unless otherwise indicated.

### General Information for Preparation Methods

Unless otherwise noted, reagents and solvents were used as received from commercial suppliers.

### EXAMPLE 1

This example depicts a general synthesis procedure for the compounds described by formula I herein. These compounds may be synthesized by one of skill in the art according to the methods shown in Johnson R W, Keenan T H, Kosh J W and Sowell J W, Synthesis of Substituted 2-Aminopyrrole Analogs of Lidocaine II, Journal of Pharmaceutical Sciences 68:955-8 (1979).

For example, the general general synthesis procedure provided by this publication is as follows.

Step 1: Preparation of 2-chloroacetamido -3-carbamyl-4,5-dimethylpyrrole (VIa). A suspension of 2-chloroacetamido -3-cyano-4,5-dimethylpyrrole (Va) (15.0. g, 0.07 mole) (1) in 100 ml of 85% phosphoric acid was stirred at room temperature for 5 min.

The vessel was placed into an oil bath preheated to 120° and stirred vigorously for 10 min. During this time, solution was achieved, and some of the product precipitated on the stirring solution surface. The vessel was removed from the oil bath and cooled to room temperature. The resulting semisolid was diluted with 300 ml of water and stirred for 10 min.

Step 2: The crude product was collected by filtration, washed with water, and air dried. One gram of the pale-yellow product (15.6 g, 96.9%) was recrystallized twice from absolute methanol to yield yellow needle-shaped crystals, which was 2-chloroacetamido -3-carbamyl-4,5-dimethylpyrrole (VIa).

Step 1: Preparation of 2-diethylaminoacetamido-3-carbamyl-4,5-dimethylpyrrole (VIIa). A suspension of VIa (9.2 g, 0.04 mole) in 125 ml of methanol and diethylamine (30 g, 0.4 mole) was refluxed, with stirring, until complete solution was achieved (0.5-3 hr). Then the solution was refluxed for an additional 1 hr, the excess diethylamine and solvent were removed in vacuo, and the residue was dissolved in 100 ml of 10% HCl.

Step 2: The solution was filtered and poured over 300 g of crushed ice. The amine was precipitated by the addition of 5% aqueous NaOH. The solid was collected by filtration, washed with distilled water, and air dried. The crude product (9.09 g, 98.9%) was recrystallized from methanol-water (4:1) to yield fine off white crystals (mp 160.5-162°) (2-diethylaminoacetamido-3-carbamyl-4,5-dimethylpyrrole (VIIa)), which were suitable for hydrochloride salt formation.

### EXAMPLE 2

This example depicts the chemical and structural analysis (e.g., NMR analysis) results of one compound (MS23) described by formula I herein, which was synthesized according to the general procedures of Example 1.

MS23 has the chemical formula of C₁₇H₃₀N₄O₂S and a molecular weight of 354.512. MS23 has an ultraviolet absorption of 307 nm in water. MS23's melting point is 110-112° C.

NMR and MS Analysis:

Proton nuclear magnetic resonance spectra were obtained on a Varian Mercury 400, a Varian Mercury 300 or a Bruker DRX 300 spectrometer. Chemical shifts are given in ppm (d) and coupling constants, J, are reported in Hertz. Internal standard was used with respect to tetramethylsilane (TMS) for proton spectra and the solvent peak was used as the reference peak for carbon spectra. Mass spectra were obtained on a Waters Micromass Quattro LC Triple quadrupole mass spectrometer equipped with an electrospray ionization (ESI) source.

Analysis of MS23 shows: ¹H NMR (CDCl₃): δ 0.89 ppm (t, 3H, J=8.4 Hz), 1.11 ppm (t, 3H, J=6.8 Hz), 1.23 ppm (d, 3H, J=6.8 Hz), 1.52 ppm (m, 2H), 2.10 ppm (s, 3H), 2.19 ppm (s, 3H), 2.48 ppm (m, 4H), 2.64 ppm (t, 2H, J=6.8 Hz), 2.78 ppm (t, 2H, J=7.2 Hz), 3.51 ppm(q, 1H, J=6.8 Hz), 5.64 ppm (s, 2H), 7.26 ppm (CDCl₃), 10.65 ppm (s, 1H), 11.86 ppm (s, 1H).

### EXAMPLE 3

Vascular Smooth Muscle Relaxation

This example illustrates the effect the compounds of the present invention have on smooth muscle relaxation. In this example, vascular smooth muscle relaxation was tested in vitro. The prototype of the compounds (“MS23”) was tested for activity to relax vascular smooth muscles using abdominal aorta and vein preparations from Sprague-Dawley rats and Sus-Scrosa pigs. The porcine tissues were obtained within 30 minutes after the pig was sacrificed. The vessels were cleaned of adherent fat and connective tissue and then cut into 3-5 mm ring segments. The segments were cut longitudinally into arterial strips without removal of the endothelium.

The strips were tied to the rat's extremities by two silk threads and vertically mounted in a water-jacketed tissue bath, maintained at 37° C., between moveable and fixed ends of a stainless steel wire. The moveable end of the wire was attached to a TRI 201 isometric transducer (LSi LETICA Scientific Instruments) which was coupled to a bridge amplifier (PowerLab, ADInstruments) for recording isometric force responses. The data was digitized through a 16 SP interface and acquired on-line with Chart 4.0 software (PowerLab ADInstruments) and stored to a personal computer.

The bath was filled with 30 mL of oxygenated (95% O₂ and 5% CO₂) Kreb solution containing, in mM, 135 NaCl, 15 NaHCO₃, 5.4 KCl, 1.2 NaH₂PO₄, 1.2 MgSO₄, 1.8 CaCl₂, and 10 glucose. The strips were equilibrated for at least 30 minutes before approximately 0.3 g (for rat tissues) or 1 g (for pig tissues) of passive tension was placed on them. Thereafter, contracture (static muscle shortening) was produced with potassium chloride by substituting 64.6 mM KCl for the same amount of NaCl in the Kreb solution (i.e., final concentration of K⁺=70 mM).

When the potassium-induced contracture reached a stable plateau level, the concentration-responsive curve of the testing compound was then recorded. Each concentration of the compound was allowed to elicit its maximal relaxation, and then a higher concentration of the compound was added to the bath solution accumulatively. The relaxation response produced by the compound was normalized to the response produced by 10 μM nitroglycerin added to the bath solution at the end of the experiment.

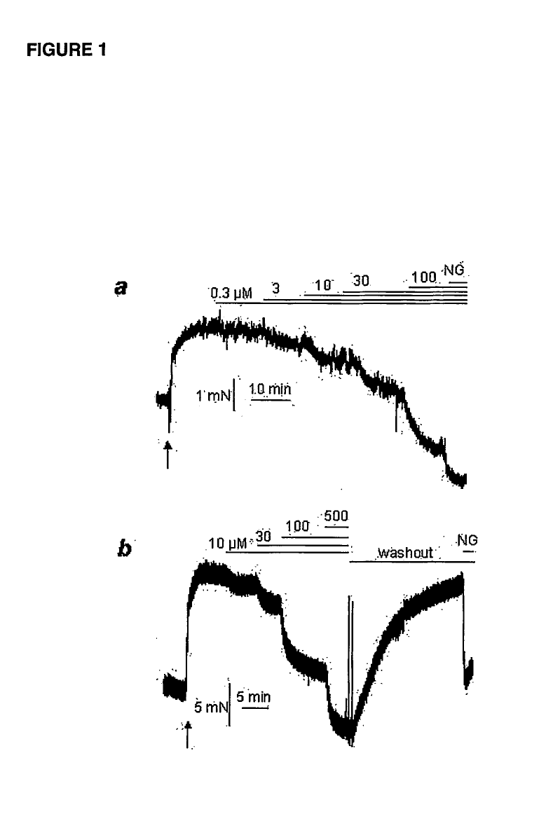

FIG. 1 shows the typical vascular relaxation responses produced by the prototype of the compounds in both arterial (panel a) and vein (panel b) tissues.

### EXAMPLE 4

Bronchial Smooth Muscle Relaxation

This example illustrates the effect the compounds of the present invention have on smooth muscle relaxation. In this example, bronchial smooth muscle relaxation was tested in vitro. MS23 was tested for activity to relax bronchial smooth muscles using the main bronchia from Sprague-Dawley rats.

The rat bronchial tissues were obtained immediately after the rat was sacrificed for another research project (Animal protocol 1091, approved by IACUC of the University of South Carolina). Both the right and left main bronchi were cleaned of adherent fat and connective tissue and trimmed into approximately 3 mm ring segments. The ring segments were then cut longitudinally at the circumference of the hyaline cartilage without removal of the endothelium. The strips, with the membranous wall (a fibrous membrane containing smooth muscular fibers) in the middle, were tied to the extremities by two silk threads and vertically mounted in a water-jacketed 37° C. tissue bath. The experimental protocol and data acquisition were same as that used for rat vascular smooth muscles (see above).

### EXAMPLE 5

Blood Pressure Reduction on Anesthetized Rats via Bolus Injection

This example illustrates the effect that MS23 has on blood pressure reduction through an in vivo experiment. Specifically, this example illustrates the effect of an intravenous bolus injection of MS23 on blood pressure. Female Sprague-Dawley rats weighing 200-225 g were anesthetized with pentobarbital (30 mg/kg body weight (bw), i.p.). The animals were positioned supinely on a heat plate that was controlled by an automatic temperature controller system (TC-324B, Warner Instrument Corp.) to maintain its body temperature constant (36° ±0.5° C.) during the experiment. The trachea, right carotid artery, and left femoral vein were exposed. The trachea was cannulated and the animal was breathed with air through a respirator at a rate of 65-70/min and an inspiration pressure of approximately 10 cm H₂O. PE catheters were implanted into the carotid artery and femoral vein, respectively. The animal was heparinized by i.v. injection of approximately 300 units of heparin dissolved in 0.3 ml of 0.9% saline. Arterial pressure was recorded from the carotid artery catheter connected to a physiological pressure transducer (SP 844, Capto Company, Norway) that was coupled to a bridge amplifier (PowerLab, ADInstruments). The data was digitized through a 16 SP interface and acquire on-line with Chart 4.0 software (PowerLab, ADInstruments) and stored to a personal computer.

After surgery, the animal was allowed to equilibrate for approximately 30 minutes before administration of the test compounds. The experiments in which the control carotid pulse pressure (difference between the systolic and diastolic pressure) was less than 30 mmHg were discarded without testing the drug's effects. Various doses (0.1-4.0 mg/kg.bw) of the test agents, dissolved in 0.9% saline, were administered as boluses through the femoral vein cannula in volumes ranging from 0.25-0.3 ml. The same volume of saline was used to flush the injection line following each administration of drug. Each dose of the compound was allowed to elicit its maximal effects. The changes in systolic, diastolic, and mean pressure (calculated as diastolic pressure ±⅓ pulse pressure) caused by the test agents were compared to that measured at the end of the 30 minute equilibration. During the experimental period, ECG was simultaneously recorded with a Bio Amplifier (PowerLab ADInstruments) through two electrodes that were placed on the right and left forearms to monitor the possible effect of the test agents on heart rate.

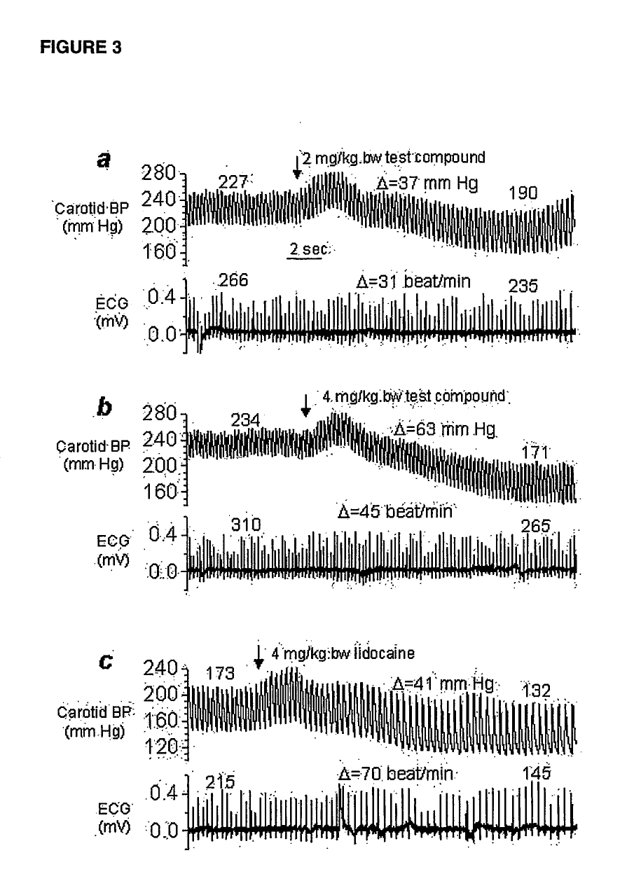

FIG. 3 shows the representative responses of blood pressure and heart rate after bolus injection of MS23 (note: the data shown in FIG. 3 were from the same rat. The lower mean blood pressure (MBP) and heart rate at the beginning of recordings in Panel C were due to application of verapamil and nitroglycerin before lidocaine injection).

### EXAMPLE 6

Blood Pressure Reduction on Anesthetized Rats via Oral Administration

This example illustrates the effect that MS23 has on blood pressure reduction through an in vivo experiment in rats. Specifically, this example illustrates the effect of an oral administration of MS23 on blood pressure.

MS23 was administered to two female rats via oral gavage at a dose of 26 and 80 mg/kg.bw, respectively. The agent was dissolved in 0.9% saline in a volume of 0.5 ml. The same volume of saline alone was administered to two control animals. After 15 minutes, the animal was anesthetized with pentobarbital (30 mg/kg.bw, i.p.) and same surgery was performed except that the femoral vein was not exposed and there was no i.v. administration of the test agent. The carotid blood pressure and heart rate were monitored and recorded for 60 minutes using the same method described above. The initial recording was made within 45 to 65 minutes after performing oral gavage. The results are reported in the following Table 1 (the MBP and HR were measured within 30 minutes after the surgery).

TABLE 1
Oral Gavage MS23 on Blood Pressure and Heart Rate

| | | MS23 | MS23 |
|---|---|---|---|
| | | 26 mg/ | 80 mg/ |
| | Saline | kg · bw | kg · bw |
| Mean Blood Pressure (mm Hg) | 186 | 156 | 126 |
| Heart Rate (beat/min) | 364 | 384 | 300 |

### EXAMPLE 7

Blood Pressure Reduction on Anesthetized Dogs via Bolus Injection

This example illustrates the effect that MS23 has on blood pressure reduction through an in vivo experiment in dogs. An intravenous bolus injection of MS23 was administered to anesthetized dogs at a dose of 0.25 mg/kg.bw. The dogs had a reduction in blood pressure of 4 mmHg. Higher doses of MS23 produced a greater decrease in blood pressure without affecting heart rate and left ventricular end diastolic pressure. See Table 2.

TABLE 2
Blood Pressure Effect in Anesthetized Dogs.

| | | | | | | Left |
|---|---|---|---|---|---|---|
| | | | | | Left | Ventric- |
| | | | | | Ventric- | ular |
| | | Blood | Blood | Blood | ular | End |
| | Heart | Pressure | Pressure | Pressure | Systolic | Diastolic |
| | Rate | Max | Min | Mean | Pressure | Pressure |
| Control | 119.8 | 99.8 | 46.6 | 67.2 | 78.5 | 8.7 |
| Bolus 1 | 118.2 | 90.5 | 39.8 | 58.4 | 71.8 | 9.0 |
| mg/kg · bw | | | | | | |
| Bolus 2 | 109.7 | 76.8 | 33.7 | 48.0 | 63.1 | 9.2 |
| mg/kg · bw | | | | | | |

### EXAMPLE 8

Relaxation Effect on Blood Vessel Rings

This example illustrates the effect the compounds of the present invention have on blood vessel ring relaxation.

The trachea, main bronchi and vessel rings (2-3 mm in width) of Sprague-Dawley rats, Harley Guinea Pigs and porchine were used to carry out the tension assay. The tissue rings were bathed in 3.5 ml or 30 ml tissue chamber and aired with 95% O₂/5% CO₂ at 37° C. The tension was measured using a tension transducer linked to a PowerLab system (ADInstruments, USA). The basal tension was set to 0.7-1.0 g step by step with a 0.1-0.2 g increment. After the base tension reached steady-state in control K-H solution, the rings were contracted for 5-10 minutes by a 70 mM high K⁺ K-H solution and relaxed by switching back to normal K-H solution. The alternation between 70 mM K⁺ K-H and normal K-H was repeated two more times to equilibrate the tissue before the experiment.

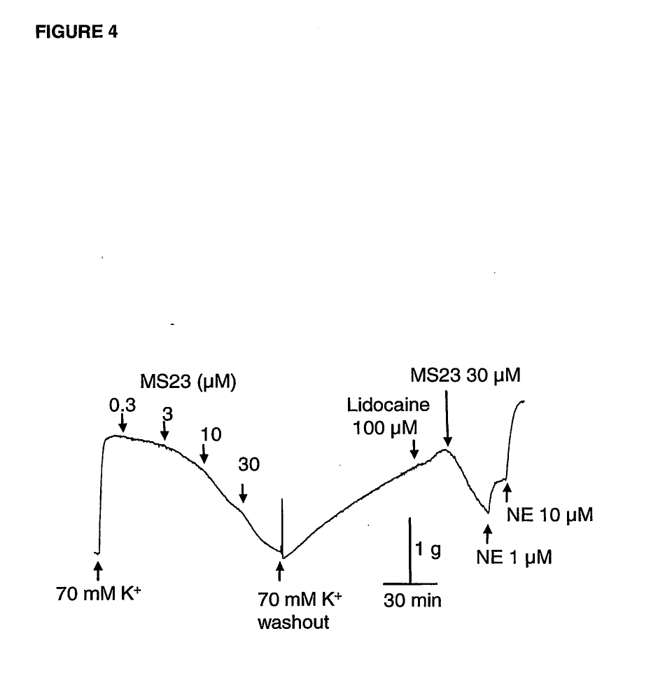

MS23 fully relaxed high K⁺-contracted (70 mM) aorta rings of rats and guinea pigs. The relaxation was also effective when tested in intra-renal and coronary arterial rings (approximately 1-2 mm) as well as pig main kidney arterial and venous rings. The vasodilatation responses were endothelium independent, reversible upon washout, and lack of desensitization. The effects were not affected by elevating extracellular K⁺ concentration from 70 mM to 140 mM. The efficacy and potency were similar in arterial and venous vessels, with a K_{d} value of approximate 15 μM as shown in FIG. 4. Note that the relaxation is reversible.

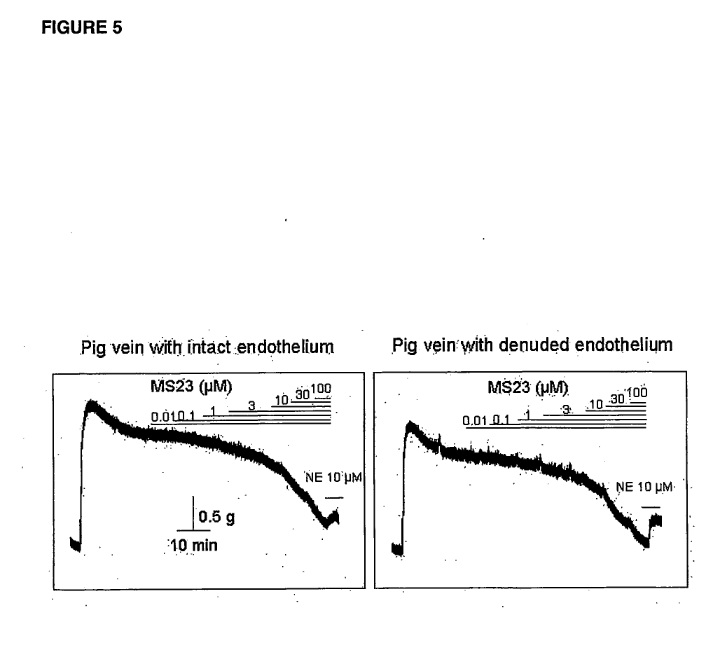

The tension assay trace shows a typical vessel ring relaxation action of MS23. Relaxation of high K⁺ contracted porcine kidney main artery by MS23 is concentration-dependent and reversible upon washout and repeatable. Lidocaine did not relax the vessel and β-adrenergic agonist Norepinephrine (NE) contracted the preparation. The relaxation effect is endothelium-independent as shown in FIG. 5.

### EXAMPLE 9

Phosphodiesterase (PDE) Assay

This example illustrates the inhibitory activity of MS23 on cAMP-specific phosphodiesterase enzymes.

Total protein was extracted from guinea pig brain tissue at 4° C. Tissues were homogenized in a buffer consisting of (in mM): Tris 20, sodium acetate 50, EGTA 2, 0.5% (v/v) and protease inhibitor cocktail (sigma) at pH 6.5. The homogenate was filtered through sterile gauze, sonicated (4×20 s at 65-70 Watts), and centrifuged at 39800 g for 20 minutes. The supernatant was used for the PDE assay. The assay buffer consisted of (in mM) 40 Tris-HCl and 10 magnesium acetate (pH 7.5) with 50 mM cAMP (10,000 cpm H³-cAMP in each tube), diluted protein extract, and inhibitors added in the reactant volume of 200 μl. The reaction was incubated at 30° C. for 30 minutes and stopped by heating in boiling water for 30 seconds. Snake venom (10 μg, Crotaius atrox, containing 5′-nucleotidase in excess) was used to convert the reaction product to adenosine or guanosine (at 30° C. for 10 minutes). AG-1-X8 anion exchange columns (Bio-Rad) were used to elute the nucleoside. The elution was collected into scintillation vials and radioactivity (in cpm) was counted using a liquid scintillation analyzer (TRI-CARB 2900IR, Packard, USA).

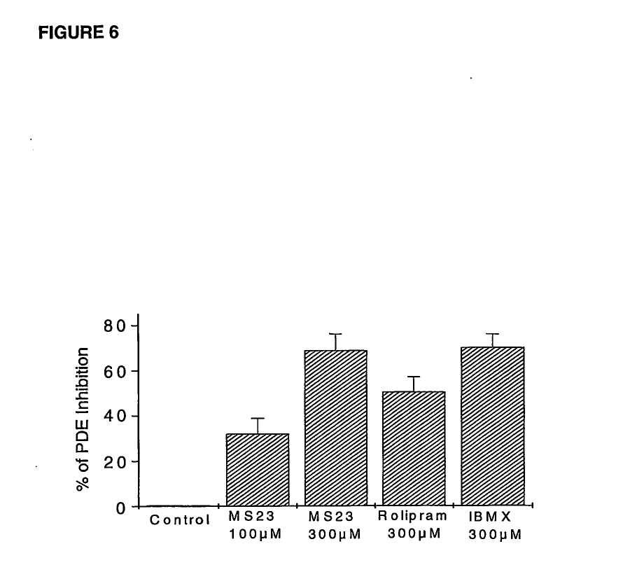

MS23 concentration-dependently inhibited the total activity of cAMP-specific phosphodiesterases (PDEs) in the protein extract from brain tissue and airway smooth muscles as shown in FIG. 6.

### EXAMPLE 9

Blood Pressure Effects in Awake Animals

This example illustrates the blood pressure effects in awake rats.

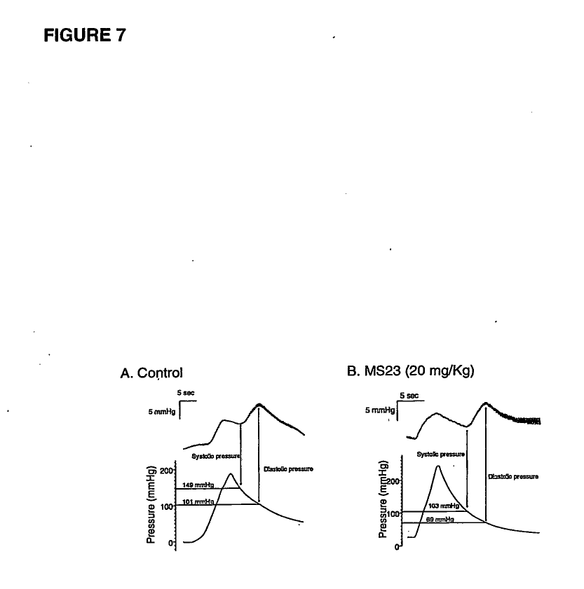

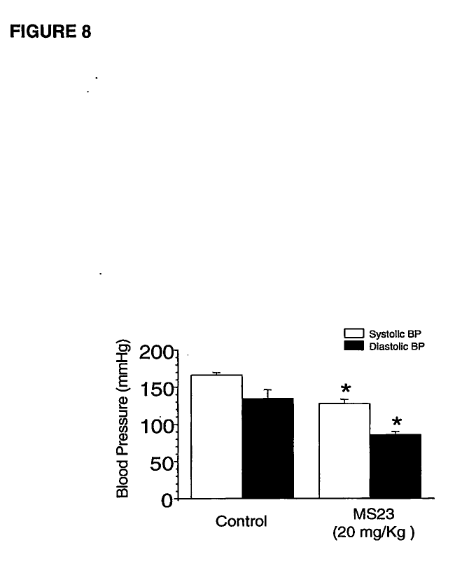

Administration of MS23, via oral gavage (20 mg/kg.bw) or intraperitoneal injection (1 mg/kg.bw), to awake SHR rats also significantly lowered blood pressure measured with non-invasive tail-cuff method as depicted in FIGS. 7 and 8. The measurements were carried out after administering the agent.

### EXAMPLE 10

Electrophysical Effects in Guinea Pig Atrial and Ventricular Myocytes

This example illustrates the electrophysical effects in guinea pig atrial and ventricular myocytes.

The effects of a novel synthesized cAMP specific phosphodiesterase inhibitor MS23 on action potential parameters were examined and compared with those of milrinone and rolipram in isolated guinea-pig myocytes. Action potentials (AP) were recorded under nystatin-perforated whole-cell patch-clamp mode at a temperature of 35±0.5° C. MS23 (30 μM) significantly shortened APD90 (action potential duration measured at 90% of repolarization) from 304.9±24.0 msec to 254.9±28.4 msec (P<0.05, n=8) in ventricular cells but had no effect on APD in atrial cells. MS23 (from 1 to 100 μM) did not prolong atrial or ventricular APD. In comparison, rolipram (30 μM) shortened APD90 from 318.3±8.9 to 274.6±9.8 msec (P<0.05, n=3) in ventricular cells. In atrial cells, rolipram produced a small delay of phase 1 and plateau phase, but acceleration of phase 3 repolarization. In contrast, milrinone (10 μM) elongated ventricular APD90 from 287.9±10.3 msec to 327.8±17.4 msec (P<0.05, n=4) and produced a significant delay of phase 1 and plateau phase, but acceleration of phase 3 repolarization (APD90 shortened from 153.9±12.1 to 135.5±12.3 msec, P<0.05, n=4) in atrial cells. After treatment of ventricular cells with either rolipram or MS23, milrinone (10 μM) markedly shortened APD90. The results indicate that MS23 may selectively affect a cAMP-specific PDE subtype that has a high enzymatic activity in ventricular myocardium.

Single myocytes were isolated by enzymatic digestion of guinea pig hearts. The animal was anesthetized by I.P. injection of pentobarbital sodium (50 mg/kg) and the heart was excised and retrogradely perfused with oxygenated Tyrode solution containing (in nM): 140 NaCl, 5.4 KCl, 1.5 MgCl₂, 1.8 CaCl₂, 0.33 NaH₂PO₄, 5.5 Glucose, 19 Hepes (pH 7.4 with NaOH at 36.5° C.). The enzymatic digestion was carried out with Ca²⁺-free Tyrode solution by adding 0.4 mg/ml of collagenase type II (306 units/mg) and 1.6 mg/ml Bovine Serum Albumin V.

Transmembrane potentials were recorded under nystatin-perforted whole-cell current-clamp mode at 36.5° C. when the cells were bathed in Tyrode solution. The finished pipette had resistance of 4-7 MΩ when filled with a perforate solution containing (in mM): 140 KCl, 2 MCl₂, 0.5 CaCl2, 10 Glucose, 10 Hepes (pH 7.2 with KOH), and 8.64 nM nystatin. The correction of junction potential was carried out off-line. The data acquisition was performed with an Axonpatch-200B amplifier/Digidata 1322A/Clampex 8.2 system (Axon Instrument, Inc., USA). Action potential was evoked by consecutive 10 msec rectangular current pulses at an interval of 2 seconds. APD was measured at 90% of repolarization (APD⁹⁰). The cell was held at a −40 mV membrane potential to inactivate the sodium channels. L-type calcium current was elicited by a step pulse from −40 mV to 0 mV with a duration of 300 msec.

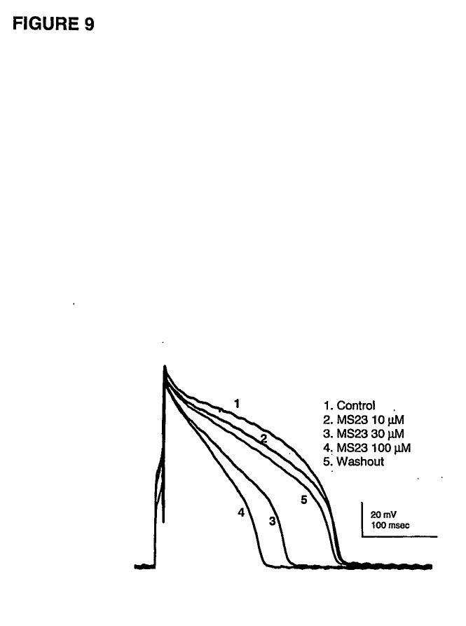

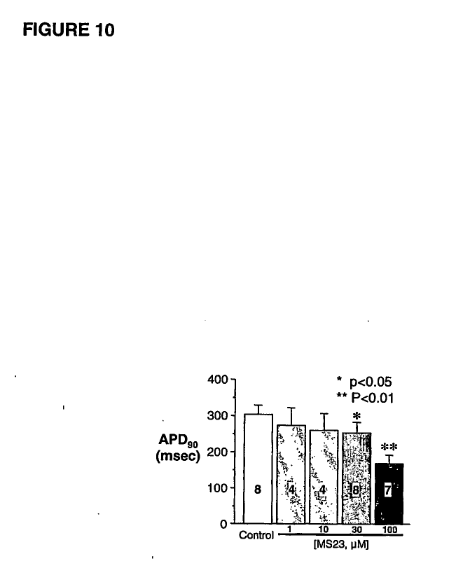

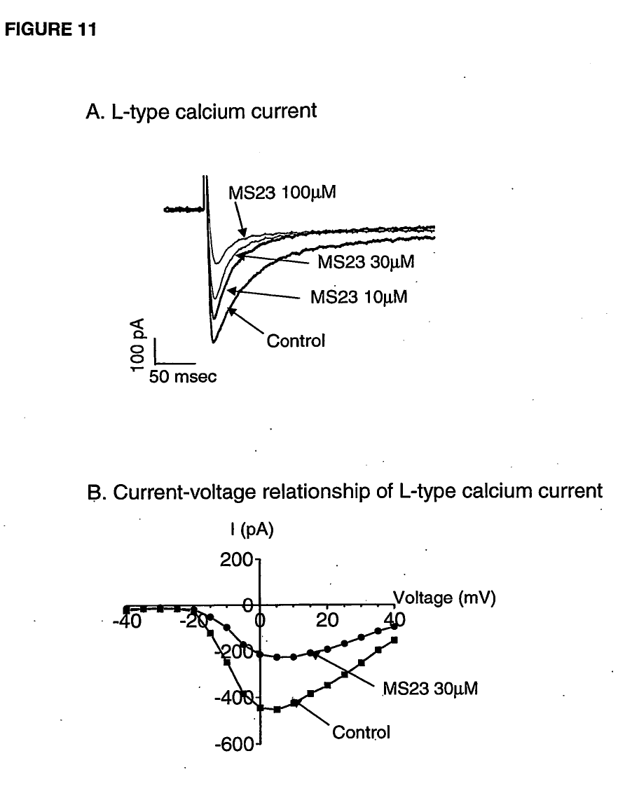

MS23 concentration-dependently reduced the amplitude of L-type calcium current. All the values were expressed as mean±SE and the differences were considered significant at *p<0.05 or **p<0.01 as depicted in FIGS. 9-12.

The L-type calcium current was recorded from a guinea pig ventricular myocyte. The cell was held at a −40 mV membrane potential to inactivate the sodium channels. L-type calcium current was elicited by a step pulse from −40 mV to 0 mV with a duration of 300 msec. MS23 concentration-dependently reduced the amplitude of L-type calcium current.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A compound having the structure: 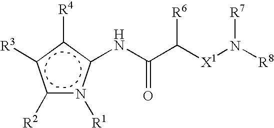
wherein:
R¹ and R³ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, alkyl, alkenyl, alkynyl, —CONR⁵R⁹, alkyl-CONR⁵R⁹, cycloalkyl, aryl, aralkyl, hydroxyalkyl, guanidinoalkyl, carboxy-R¹⁰, hydroxyaralkyl, alkoxyalkyl, aminoalkyl, alkylamino-R¹⁰, thioalkyl, alkylthio-R¹⁰, alkylsulfonyl-R¹⁰, alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, alkyl, —CONR⁵R⁹, alkyl-CONR⁵R⁹, alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, carbamyl, alkylcarbamyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl;
with the proviso that when R⁶ is hydrogen, R⁷ is other than ethyl or X¹ is other than methyl, when R⁷ is ethyl, R⁶ is other than hydrogen or X¹ is other than methyl, and when X¹ is methyl, R⁶ is other than hydrogen or R⁷ is other than ethyl; and
including the isomers, racemates, salts, and prodrugs thereof.

2. The compound according to claim 1, wherein:
R¹ and R³ are independently selected from —H, C₁-C₆ alkyl, C₁-C₆ alkenyl, C₁-C₆ alkynyl, —CONR⁵R⁹, alkyl-CONR⁵R⁹, C₁-C₆ alkylthio-R¹⁰, thio-(C₁-C₆) alkyl, amino-(C₁-C₆) alkyl, C₁-C₆ alkylamino-R¹⁰, cycloalkyl, aryl, and aralkyl, wherein R¹ and R³ are independently substituted or unsubstituted, which if substituted, are substituted with one or more substituents selected from R¹¹;
R² is selected from —H, C₁-C₆ alkyl, C₁-C₆ alkenyl, C₁-C₆ alkynyl, —CONR⁵R⁹, alkyl-CONR⁵R⁹, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰, wherein R² is substituted or unsubstituted, which if substituted, is substituted with one or more substituents selected from R¹¹;
R⁴ is selected from —H, cyano, C₁-C₆ alkyl, —CONR⁵R⁹, alkyl-CONR⁵R⁹, C₁-C₆ alkylsulfonyl-R¹⁰, alkylsulfinyl-R¹⁰, alkylthio-R¹⁰, and alkylamino-R¹⁰;
R⁵ and R⁹ are independently selected from —H, alkyl, alkenyl, alkynyl, alkylthio-R¹⁰, thioalkyl, aminoalkyl, alkylamino-R¹⁰, cycloalkyl, aryl, aralkyl, wherein R⁵ and R⁹ are independently substituted or unsubstituted, which if substituted, are substituted with one or more substituents selected from R¹¹;
R⁶ and R¹⁰ are independently selected from —H and alkyl;
R⁷ and R⁸ are independently selected from —H, alkyl, alkenyl, alkynyl, —CONR⁵R⁹, alkyl-CONR⁵R⁹, alkylthio-R¹⁰, thioalkyl, aminoalkyl, alkylamino-R¹⁰, cycloalkyl, aryl, aralkyl, wherein R⁷ and R⁸ are independently substituted or unsubstituted, which if substituted, are substituted with one or more substituents selected from R¹¹;
R¹¹ is selected from halo and haloalkyl;
X¹ is optionally present, and if present, is alkyl;
with the proviso that when R⁶ is hydrogen, R⁷ is other than ethyl or X¹ is other than methyl, when R⁷ is ethyl, R⁶ is other than hydrogen or X¹ is other than methyl, and when X¹ is methyl, R⁶ is other than hydrogen or R⁷ is other than ethyl; and
including the isomers, racemates, salts, and prodrugs thereof.

3. The compound according to claim 1, wherein:
R¹ and R³ are independently selected from —H, C₁-C₆ alkyl, C₁-C₆ alkenyl, C₁-C₆ alkynyl, carbamyl, carbamylalkyl, C₁-C₆ alkylthio-R¹⁰, thio-(C₁-C₆) alkyl, amino-(C₁-C₆) alkyl, C₁-C₆ alkylamino-R¹⁰, cycloalkyl, aryl, and aralkyl, wherein R¹ and R³ are independently substituted or unsubstituted, which if substituted, are substituted with a halo substituent;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰, wherein R² is independently substituted or unsubstituted, which if substituted, is substituted with a halo substituent;
R⁴ is carbamyl;
R⁶ is selected from —H and C₁-C₆ alkyl;
R⁷ and R⁸ are independently C₁-C₆ alkyl;
X¹ is optionally present, and if present, is C₁-C₄ alkyl;
with the proviso that when R⁶ is hydrogen, R⁷ is other than ethyl or X¹ is other than methyl, when R⁷ is ethyl, R⁶ is other than hydrogen or X¹ is other than methyl, and when X¹ is methyl, R⁶ is other than hydrogen or R⁷ is other than ethyl; and
including the isomers, racemates, salts, and prodrugs thereof.

4. The compound according to claim 1, wherein:
R¹ and R³ are independently selected from —H, C₁-C₆ alkyl, carbamyl, carbamylalkyl, C₁-C₆ alkylthio-R¹⁰, and C₁-C₆ alkylamino-R¹⁰;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R⁴ is carbamyl;
R⁶ is selected from —H and C₁-C₄ alkyl;
R⁷ and R⁸ are independently selected from C₁-C₄ alkyl;
X¹ is absent;
with the proviso that when R⁶ is hydrogen, R⁷ is other than ethyl, when R⁷ is ethyl, R⁶ is other than hydrogen; and
including the isomers, racemates, salts, and prodrugs thereof.

5. The compound according to claim 1, wherein:
R¹ and R³ are independently selected from —H, C₁-C₆ alkyl, —CONR⁵R⁹, C₁-C₆ alkylthio-R¹⁰, and C₁-C₆ alkylamino-R¹⁰;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R⁴ is carbamyl;
R⁶ is selected from —H and C₁-C₄ alkyl;
R⁷ and R⁸ are independently selected from ethyl and propyl;
X¹ is absent;
with the proviso that when R⁶ is hydrogen, R⁷ is other than ethyl, when R⁷ is ethyl, R⁶ is other than hydrogen; and
including the isomers, racemates, salts, and prodrugs thereof.

6. The compound according to claim 1, wherein:
R¹ is —H;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R³ is methyl;
R⁴ is carbamyl;
R⁶ is selected from —H and methyl;
R⁷ and R⁸ are independently C₁-C₄ alkyl;
X¹ is absent;
with the proviso that when R⁶ is hydrogen, R⁷ is other than ethyl, when R⁷ is ethyl, R⁶ is other than hydrogen; and
including the isomers, racemates, salts, and prodrugs thereof.

7. The compound according to claim 1, wherein:
R¹ is —H;
R² is selected from —H, C₁-C₆ alkyl, C₁-C₆ alkenyl, C₁-C₆ alkynyl, carbamyl, carbamylalkyl, cycloalkyl, aryl, aralkyl, hydroxy-(C₁-C₆) alkyl, guanidino-(C₁-C₆) alkyl, carboxy-R¹⁰, hydroxyaralkyl, alkoxyalkyl, amino-(C₁-C₆) alkyl, C₁-C₆ alkylamino-R¹⁰, thio-(C₁-C₆) alkyl, C₁-C₆ alkylthio-R¹⁰, C₁-C₆ alkylsulfonyl-R¹⁰, C₁-C₆ alkylsulfinyl-R¹⁰, heteroaryl, heteroaryl-R¹⁰, heterocyclyl, and heterocyclyl-R¹⁰;
R³ is methyl;
R⁴ is carbamyl;
R⁶ is methyl;
R⁷ and R⁸ are independently C₁-C₄ alkyl;
X¹ is absent; and
including the isomers, racemates, salts, and prodrugs thereof.

8. The compound according to claim 1, wherein:
R¹ is —H;
R² is selected from —H, C₁-C₄ alkyl, carbamyl, C₁-C₄ alkylamino-R¹⁰, C₁-C₄ alkylthio-R¹⁰;
R³ is methyl;
R⁴ is carbamyl;
R⁶ is methyl;
R⁷ and R⁸ are independently selected from ethyl and propyl;
X¹ is absent; and
including the isomers, racemates, salts, and prodrugs thereof.

9. The compound according to claim 1, wherein:
R¹ is —H;
R² is selected from —H, C₁-C₄ alkyl, carbamyl, C₁-C₄ alkylamino-R¹⁰, C₁-C₄ alkylthio-R¹⁰;
R³ and R⁶ are methyl;
R⁴ is carbamyl;
R⁷ and R⁸ are independently selected from ethyl and propyl;
X¹ is absent; and
including the isomers, racemates, salts, and prodrugs thereof.

10. The compound according to claim 1, wherein:
R¹ is —H;
R² is selected from —H, methyl, ethyl, carbamyl, dimethylthio, methylthioethyl, ethylthiomethyl, diethylthio, dimethylamino, and methylaminoethyl;
R³ and R⁶ are methyl;
R⁴ is carbamyl;
R⁷ and R⁸ are independently selected from ethyl and propyl;
X¹ is absent; and
including the isomers, racemates, salts, and prodrugs thereof.

11. The compound according to claim 1, wherein:
R¹ is —H;
R² is selected from —H, dimethylthio, methylthioethyl, ethylthiomethyl, and diethylthio;
R³ and R⁶ are methyl;
R⁴ is carbamyl;
R⁷ is propyl;
R⁸ is ethyl;
X¹ is absent; and
including the isomers, racemates, salts, and prodrugs thereof.

12. The compound according to claim 1, wherein the compound comprises the structure: 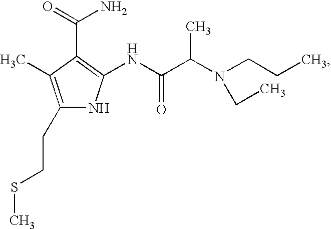
including the isomers, racemates, salts, and prodrugs thereof.

13. The compound according to claim 1, wherein the compound comprises 2-[2-(N-ethyl-N-n-propyl) amino] propionamido-3-carbamyl-4-methyl-5-(methylthio) pyrrole.

14. The compound according to claim 1, wherein the compound comprises a dual PDE-4/Ca²⁺-channel inhibitor.

15. A therapeutic composition comprising a compound having a structure described in claim 1.

16. A pharmaceutical composition comprising a pharmaceutically acceptable excipient and at least one compound having a structure described in claim 1.

17. A kit comprising a dosage form that includes a therapeutically effective amount of at least one compound comprising a structure described in claim 1.

18. A method of inhibiting a phosphodiesterase enzyme, the method comprising contacting the phosphodiesterase enzyme with at least one compound having a structure described in claim 1.

19. The method according to claim 18, wherein the phosphodiesterase enzyme comprises a phosphodiesterase-4 enzyme.

20. The method according to claim 18, wherein the phosphodiesterase enzyme comprises a phosphodiesterase-3 enzyme.

21. A method of inhibiting L-type calcium channels, the method comprising contacting an L-type calcium channel with at least one compound having a structure described in claim 1.

22. A method of preventing or treating a cardiovascular or respiratory disorder in a subject, the method comprising administering to the subject an effective amount of a compound having a structure described in claim 1.

23. The method according to claim 22, wherein the compound is a phosphodiesterase inhibitor.

24. The method according to claim 23, wherein the compound is a cAMP-specific phosphodiesterase inhibitor.

25. The method according to claim 23, wherein the compound is a selective phosphodiesterase inhibitor.

26. The method according to claim 24, wherein the compound is a selective phosphodiesterase-4 inhibitor.

27. The method according to claim 24, wherein the compound is a phosphodiesterase-3 inhibitor.

28. The method according to claim 26, wherein the selective phosphodiesterase-4 inhibitor has an IC₅₀ for inhibition of phosphodiesterase-3 of greater about 60 μM.

29. The method according to claim 28, wherein the phosphodiesterase-4 inhibitor provides an IC₅₀ of less than about 200 μM.

30. The method according to claim 28, wherein the phosphodiesterase-4 inhibitor provides an IC₅₀ of less than about 50 μM.

31. The method according to claim 28, wherein the phosphodiesterase-4 inhibitor provides an IC₅₀ of less than about 5 μM.

32. The method according to claim 28, wherein the phosphodiesterase-4 inhibitor provides an IC₅₀ of about 2 μM.

33. The method according to claim 22, wherein the subject is one that is in need of the prevention or treatment of a cardiovascular or respiratory disorder.

34. The method according to claim 22, wherein the cardiovascular disorder is chosen from myocardial ischemia, transient ischemic attack, hypertension, hypotension, heart arrhythmias, including atrial fibrillation and flutter, tachycardia, and ventricular fibrillation, pulmonary hypertension, hypokalemia, angina pectoris, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis arteriosclerosis, atherosclerosis, calcification (such as vascular calcification and valvar calcification), coronary artery disease, coronary heart disease, peripheral arterial disease, heart failure, congestive heart failure, shock, stroke, left ventricular hypertrophy, angina, diabetic nephropathy, kidney failure, eye damage, cardiac damage, diabetic cardiac myopathy, renal insufficiency, renal injury, renal arteriopathy, peripheral vascular disease, left ventricular hypertrophy, cognitive dysfunction, headache, aortic aneurysm, deep vein thrombosis, bacterial endocarditis, cardiomyopathy, congenital cardiovascular defects, rheumatic heart disease, valvular heart disease, Adams-Stokes disease, antiphospholipid syndrome, aortic regurgitation, long Q-T syndrome, Marfan syndrome, Raynaud's syndrome, Wolff-Parkinson-White syndrome (WPW).

35. The method according to claim 22, wherein the respiratory disorder is chosen from asthma, spasmodic asthma, bronchitis, chronic obstructive pulmonary disease (COPD), cystic fibrosis, pulmonary embolism, pneumonia, pulmonary fibrosis, respiratory failure, acute respiratory distress syndrome, bronchiectasis, rhinitis, chronic rhinitis, sinusitis, chronic sinusitis, emphysema, pulmonary sarcoidosis, tuberculosis, alpha-1 antitrypsin deficiency, allergies, alveolar capillary dysplasia, asbestosis, black lung, bronchiolitis, cold, goodpasture syndrome, laryngeal cancer, laryngomalacia, legionnaires' disease, lung cancer, lymphagioleiomyomatosis (LAM), persistent cough, pleurisy (Pleuritis), Pneumothorax, Respiratory Syncytial Virus (RSV), severe acute respiratory syndrome (SARS), silicosis, sinus infection, tonsillitis, valley fever, recurrent respiratory papillomatosis, bronchopulmonary dysplasia (BPD), influenza, hantavirus pulmonary syndrome (HPS), hayfever, primary ciliary dyskinesia (PCD), kartagener's syndrome, lymphangioleiomyomatosis (LAM), mesothelioma, primary pulmonary hypertension (PPH), spontaneous pneumothorax, meningococcemia, and wegener's granulomatosis.

36. A method of preventing or treating a cardiovascular or respiratory disorder in a subject, the method comprising administering to the subject a phosphodiesterase-4 inhibitor in combination with a calcium channel blocker, wherein the phosphodiesterase-4 inhibitor and the calcium channel blocker are the same compound which is a compound described in claim 1.

37. A method of modulating the activity of a phosphodiesterase enzyme in a subject in need of such modulation, the method comprising administering to the subject a compound comprising a structure described in claim 1.

38. A method of modulating the activity of an L-type calcium channel in a subject in need of such modulation, the method comprising administering to the subject a compound comprising a structure described in claim 1.

39. A method of modulating the activity of a phosphodiesterase enzyme and an L-type calcium channel in a subject in need of such modulation, the method comprising administering to the subject a compound having a structure described in claim 1.

40. A method of preventing or treating a respiratory disorder in a subject, the method comprising administering to the subject a compound having a structure described in claim 1 in combination with a β-adrenergic agonist.

41. The method according to claim 40, wherein the β-adrenergic agonist comprises a β₂-adrenergic agonist.

42. The method according to claim 41, wherein the β₂-adrenergic agonist comprises at least one compound chosen from metaproterenol, pirbuterol, albuterol, levalbuterol, formoterol, salmeterol, terbutaline, isoetharine, levalbuterol, salbutamol, bambuterol, fenoterol, reproterol, tulobuterol, and mixtures thereof.

43. Use of a compound having a structure described in claim 1 alone or in combination with a β-adrenergic agonist for the production of a medicament for the preventing or treating a cardiovascular or respiratory disorder in a subject.

44. A method of preventing or treating a cardiovascular or respiratory disorder in a subject, the method comprising administering to the subject an effective amount of a compound having a structure described in claim 1 in combination with a convention treatment agent.

45. The method according to claim 44, wherein the conventional treatment agent is a calcium channel blocker.
